SEQUENCE LISTING (82 sequences)

SEQ ID NO: 1 - DNA, 32 bp, Artificial Sequence
  Synthetic oligonucleotide
tttgttactgtggtagatactacnngnagnac

SEQ ID NO: 2 - DNA, 32 bp, Artificial Sequence
  Synthetic oligonucleotide
tttgttactgttgttgatactacacgcagtac

SEQ ID NO: 3 - DNA, 32 bp, Artificial Sequence
  Synthetic oligonucleotide
tttgttactgtggtagataccactcgcagtac

SEQ ID NO: 4 - DNA, 32 bp, Artificial Sequence
  Synthetic oligonucleotide
tttgttactgtggtagataccacacgtagtac

SEQ ID NO: 5 - DNA, 32 bp, Artificial Sequence; identical to SEQ ID NO: 3
  Synthetic oligonucleotide

SEQ ID NO: 6 - DNA, 32 bp, Artificial Sequence
  Synthetic oligonucleotide
tttgttactgtagttgatacaacccgtagtac

SEQ ID NO: 7 - DNA, 32 bp, Artificial Sequence
  Synthetic oligonucleotide
tttcttactgttgtggacactacccgtagtac

SEQ ID NO: 8 - DNA, 32 bp, Artificial Sequence
  Synthetic oligonucleotide
tttgttactgtagtggacactacccgcagtac

SEQ ID NO: 9 - DNA, 32 bp, Artificial Sequence
  Synthetic oligonucleotide
tttattacctgtgttgatactaccagaagtac

SEQ ID NO: 10 - DNA, 32 bp, Artificial Sequence
  Synthetic oligonucleotide
tttgtcacagttgtggataccactcgtagcac

SEQ ID NO: 11 - DNA, 32 bp, Artificial Sequence
  Synthetic oligonucleotide
tttgttactgtagtagatactactagaagtac

SEQ ID NO: 12 - DNA, 32 bp, Artificial Sequence
  Synthetic oligonucleotide
tttgttaccgtggttgataccactcgtagcac

SEQ ID NO: 13 - DNA, 32 bp, Artificial Sequence
  Synthetic oligonucleotide
tttttaacagttgtagatactactcgcagcac

SEQ ID NO: 14 - DNA, 32 bp, Artificial Sequence
  Synthetic oligonucleotide
tttgttactgttgtggatactaccagaagtac

SEQ ID NO: 15 - DNA, 32 bp, Artificial Sequence
  Synthetic oligonucleotide
tttcttactgttgtggataccactcgcagtac

SEQ ID NO: 16 - DNA, 24 bp, Artificial Sequence
  Synthetic oligonucleotide
gaaaaataaactgtaaatcatant

SEQ ID NO: 17 - DNA, 24 bp, Artificial Sequence
  Synthetic oligonucleotide
gaaaaataaactgtaaatcatatt

SEQ ID NO: 18 - DNA, 24 bp, Artificial Sequence
  Synthetic oligonucleotide
gaaaaataaactgcaaatcatatt

SEQ ID NO: 19 - DNA, 24 bp, Artificial Sequence
  Synthetic oligonucleotide
gaaatataaattgtaaatcaaatt

SEQ ID NO: 20 - DNA, 24 bp, Artificial Sequence
  Synthetic oligonucleotide
gaaaaacaaactgtagatcatatt

SEQ ID NO: 21 - DNA, 24 bp, Artificial Sequence
  Synthetic oligonucleotide
gaaaaataaactgtaaatcatatt

SEQ ID NO: 22 - DNA, 24 bp, Artificial Sequence
  Synthetic oligonucleotide
gaaatataaattgtaaatcatact

SEQ ID NO: 23 - DNA, 24 bp, Artificial Sequence
  Synthetic oligonucleotide
gaaaaataaactgtaaatcatatt

SEQ ID NO: 24 - DNA, 24 bp, Artificial Sequence
  Synthetic oligonucleotide
gaaaaataaattgcaattcatact

SEQ ID NO: 25 - DNA, 24 bp, Artificial Sequence
  Synthetic oligonucleotide
gaaaaataaattgtaaatcaaatt

SEQ ID NO: 26 - DNA, 24 bp, Artificial Sequence
  Synthetic oligonucleotide
gaaaaacaaattgtaattcatatt

SEQ ID NO: 27 - DNA, 24 bp, Artificial Sequence
  Synthetic oligonucleotide
gaaaaacaaactgtaagtcatact

SEQ ID NO: 28 - DNA, 24 bp, Artificial Sequence
  Synthetic oligonucleotide
gaaatataaactgcaaatcaaatt

SEQ ID NO: 29 - DNA, 24 bp, Artificial Sequence
  Synthetic oligonucleotide
gaaacacaaactgtagttcatatt

SEQ ID NO: 30 - DNA, 24 bp, Artificial Sequence
  Synthetic oligonucleotide
gaaatataaattgcaaatcatatt

SEQ ID NO: 31 - DNA, 142 bp, Human Papilloma Virus (HPV)
tttgttactgttgttgatactacacgcagtacaaatatgtcattatgtgctgccatatctacttcagaaactacatataaaaatactaactttaaagagtacctacgacatggggaggaatatgatttacagtttatttttc

SEQ ID NO: 32 - DNA, 142 bp, Human Papilloma Virus (HPV)
tttgttactgttgttgatactacacgcagtacaaatatgtcattatgtgctgccatatctacttcagaaactacatataaaaatactaactttaaggagtacctacgacatggggaggaatatgatttacagtttatttttc

SEQ ID NO: 33 - DNA, 145 bp, Human Papilloma Virus (HPV)
tttgttactgtggtagataccactcgcagtaccaatttaacaatatgtgcttctacacagtctcctgtacctgggcaatatgatgctaccaaatttaagcagtatagcagacatgttgaggaatatgatttgcagtttatttttc

SEQ ID NO: 34 - DNA, 145 bp, Human Papilloma Virus (HPV); identical to SEQ ID NO: 33

SEQ ID NO: 35 - DNA, 139 bp, Human Papilloma Virus (HPV)
tttgttactgtggtagataccactcgcagtactaatatgactttatgcacacaagtaactagtgacagtacatataaaaatgaaaattttaaagaatatataagacatgttgaagaatatgatctacagtttgtttttc

SEQ ID NO: 36 - DNA, 139 bp, Human Papilloma Virus (HPV); identical to SEQ ID NO: 35

SEQ ID NO: 37 - DNA, 142 bp, Human Papilloma Virus (HPV)
tttgttactgtagttgatacaacccgtagtacaaatatgtctgtgtgttctgctgtgtcttctagtgacagtacatataaaaatgacaattttaaggaatatttaaggcatggtgaagaatatgatttacagtttatttttc

SEQ ID NO: 38 - DNA, 142 bp, Human Papilloma Virus (HPV)
tttgttactgtagttgatacaacccgtagtacaaatatgtctgtgtgttctgctgtgtctactagtgacagtacatataaaaatgacaattttaaggaatatttaaggcatggtgaagaatatgatttacagtttatttttc

SEQ ID NO: 39 - DNA, 145 bp, Human Papilloma Virus (HPV)
tttcttactgttgtggacactacccgtagtaccaactttacattatctacctctatagagtcttccataccttctacatatgatccttctaagtttaaggaatataccaggcacgtggaggagtatgatttacaatttatatttc

SEQ ID NO: 40 - DNA, 145 bp, Human Papilloma Virus (HPV)
tttcttactgtagtggacactacccgtagtaccaactttacattatctacctctatagagtcttccataccttctacatatgatccttctaagtttaaggaatatatcaggcacgtggaggagtatgatttacaatttatatttc

SEQ ID NO: 41 - DNA, 145 bp, Human Papilloma Virus (HPV)
tttcttactgtagtggacactacccgtagtaccaactttacattatctacctctatagagtcttccataccttctacatatgatccttctaagtttaaggaatataccaggcacgtggaggagtatgatttacaatttatatttc

SEQ ID NO: 42 - DNA, 145 bp, Human Papilloma Virus (HPV)
tttgttactgtagtggacactacccgcagtactaatttaacattatgtgcctctacacaaaatcctgtgccaagtacatatgaccctactaagtttaagcagtatagtagacatgtggaggaatatgatttacagtttatttttc

SEQ ID NO: 43 - DNA, 142 bp, Human Papilloma Virus (HPV)
tttattacctgtgttgatactaccagaagtacaaatttaactattagcactgccactgctgcagtttccccaacatttactccaagtaactttaagcaatatattaggcatggggaagagtatgaattgcaatttatttttc

SEQ ID NO: 44 - DNA, 142 bp, Human Papilloma Virus (HPV)
tttattacctgtgttgatactaccagaagtacaaatttaactattagcactgccactgctgcggtttccccaacatttactccaagtaactttaagcaatatattaggcatggggaagagtatgaattgcaatttatttttc

SEQ ID NO: 45 - DNA, 139 bp, Human Papilloma Virus (HPV)
tttgtcacagttgtggataccactcgtagcactaacatgactttatgtgctgaggttaaaaaggaaagcacatataaaaatgaaaattttaaggaataccttcgtcatggcgaggaatttgatttacaatttatttttc

SEQ ID NO: 46 - DNA, 139 bp, Human Papilloma Virus (HPV)
tttgtcacagttgtggataccacccgtagcactaacatgactttatgtgctgaggttaaaaaggaaagcacatataaaaatgaaaattttaaggaataccttcgtcatggcgaggaatttgatttacaatttatttttc

SEQ ID NO: 47 - DNA, 139 bp, Human Papilloma Virus (HPV)
tttgttactgtagtagatactactagaagtactaacatgactattagtactgctacagaacagttaagtaaatatgatgcacgaaaaattaatcagtaccttagacatgtggaggaatatgaattacaatttgtttttc

SEQ ID NO: 48 - DNA, 139 bp, Human Papilloma Virus (HPV); identical to SEQ ID NO: 47

SEQ ID NO: 49 - DNA, 139 bp, Human Papilloma Virus (HPV)
tttgttaccgtggttgataccactcgtagcactaatatgacattatgcactgaagtaactaaggaaggtacatataaaaatgataattttaaggaatatgtacgtcatgttgaagagtatgacttacagtttgtttttc

SEQ ID NO: 50 - DNA, 139 bp, Human Papilloma Virus (HPV)
tttgttaccgtggttgataccactcgtagcactaatatgacattatgcactgaagtaactaaggaaggtacatataaaaatgataattttaaggaatatgtacgtcatgttgaagaatatgacttacagtttgtttttc

SEQ ID NO: 51 - DNA, 145 bp, Human Papilloma Virus (HPV)
tttttaacagttgtagatactactcgcagcaccaatctttctgtgtgtgcttctactacttcttctattcctaatgtatacacacctaccagttttaaagaatatgccagacatgtggaggaatttgatttgcagtttatatttc

SEQ ID NO: 52 - DNA, 145 bp, Human Papilloma Virus (HPV); identical to SEQ ID NO: 51

SEQ ID NO: 53 - DNA, 145 bp, Human Papilloma Virus (HPV)
tttcttactgttgtggataccactcgcagtaccaattttactttgtctactactactgaatcagctgtaccaaatatttatgatcctaataaatttaaggaatatattaggcatgttgaggaatatgatttgcaatttatatttc

SEQ ID NO: 54 - DNA, 145 bp, Human Papilloma Virus (HPV); identical to SEQ ID NO: 53

SEQ ID NO: 55 - DNA, 142 bp, Human Papilloma Virus (HPV)
  misc_feature (15)..(15): n = A, T or G
tttgttactgttgtngatactactcgcagtacnaatatgactttatgtactgctatatctactatancaagtacatataaanctantaattttaaggaatatattagacatgtggaggaatatgatttacagtttatttttc

SEQ ID NO: 56 - DNA, 21 bp, Human Papilloma Virus (HPV)
ggtagatactachmgyagyac

SEQ ID NO: 57 - DNA, 12 bp, Human Papilloma Virus (HPV)
tachmgyagyac

SEQ ID NO: 58 - DNA, 13 bp, Human Papilloma Virus (HPV)
tgtaaatcatayt

SEQ ID NO: 59 - DNA, 13 bp, Human Papilloma Virus (HPV)
aatcaatcatayt

SEQ ID NO: 60 - protein, 224 aa, Pyrococcus furiosus
MKIGGIDEAGRGPAIGPLVVATVVVDEKNIEKLRNIGVKDSKQLTPHERKNLFSQITSIADDYKIVIVSPEEIDNRSGTMNELEVEKFALALNSLQIKPALIYADAADVDANRFASLIERRLNYKAKIIAEHKADAKYPVVSAASILAKVVRDEEIEKLKKQYGDFGSGYPSDPKTKKWLEEYYKKHNSFPPIVRRTWETVRKIEESIKAKKSQLTLDKFFKKP

SEQ ID NO: 61 - protein, 16 aa, Pyrococcus furiosus
GIDEAGRGPAIGPLVV

SEQ ID NO: 62 - protein, 12 aa, Pyrococcus furiosus
LRNIGVKDSKQL

SEQ ID NO: 63 - protein, 19 aa, Pyrococcus furiosus
HKADAKYPVVSAASILAKV

SEQ ID NO: 64 - protein, 16 aa, Pyrococcus furiosus
KLKKQYGDFGSGYPSD

SEQ ID NO: 65 - protein, 166 aa, Thermus thermophilus
MNPSPRKRVALFTDGACLGNPGPGGWAALLRFHAHEKLLSGGEACTTNNRMELKAAIEGLKALKEPCEVDLYTDSHYLKKAFTEGWLEGWRKRGWRTAEGKPVKNRDLWEALLLAMAPHRVRFHFVKGHTGHPENERVDREARRQAQSQAKTPCPPRAPTLFHEEA

SEQ ID NO: 66 - protein, 25 aa, Thermus thermophilus
  MISC_FEATURE (2)..(2): Xaa = any naturally occurring amino acid
KXVXLFTDGXCXGXPGXGGXALLRY

SEQ ID NO: 67 - protein, 8 aa, Thermus thermophilus
TTNNRMEL

SEQ ID NO: 68 - protein, 5 aa, Thermus thermophilus
KPVKN

SEQ ID NO: 69 - protein, 12 aa, Thermus thermophilus
  MISC_FEATURE (6)..(6): Xaa can be any naturally occurring amino acid
FVKGHXGHXENE

SEQ ID NO: 70 - DNA, 16 bp, Human Papilloma Virus (HPV)
atactachmgyagyac

SEQ ID NO: 71 - protein, 224 aa, Pyrococcus furiosus; identical to SEQ ID NO: 60

SEQ ID NO: 72 - protein, 220 aa, Pyrococcus horikoshi
MKVAGVDEAGRGPVIGPLVIGVAVIDEKNIERLRDIGVKDSKQLTPGQREKLFSKLIDILDDYYVLLVTPKEIDERHHSMNELEAEKFVVALNSLRIKPQKIYVDSADVDPKRFASLIKAGLKYEATVIAEHKADAKYEIVSAASIIAKVTRDREIEKLKQKYGEFGSGYPSDPRTKEWLEEYYKQYGDFPPIVRRTWETARKIEERFRKNQLTLDKFLK

SEQ ID NO: 73 - protein, 228 aa, Thermococcus kodakarensis
MKIAGIDEAGRGPVIGPMVIAAVVVDENSLPKLEELKVRDSKKLTPKRREKLFNEILGVLDDYVILELPPDVIGSREGTLNEFEVENFAKALNSLKVKPDVIYADAADVDEERFARELGERLNFEAEVVAKHKADDIFPVVSAASILAKVTRDRAVEKLKEEYGEIGSGYPSDPRTRAFLENYYREHGEFPPIVRKGWKTLKKIAEKVESEKKAEERQATLDRYFRKV

SEQ ID NO: 74 - protein, 211 aa, Archaeoglobus profundus
MIAGIDEAGKGPVIGPLVICGVLCDEETVEYLKSVGVKDSKKLDRRKREELYNIIKSLCKVKVLKISVEDLNRLMEYMSINEILKRAYVEIIRSLMPKVVYIDCPDINVERFKHEIEERTGVEVFASHKADEIYPIVSIASIVAKVERDFEIDKLKKIYGDFGSGYPSDLRTIEFLRSYLREHKSFPPIVRKRWKTLKRLTTHTLSDFFEV

SEQ ID NO: 75 - protein, 205 aa, Archaeoglobus fulgidis
MKAGIDEAGKGCVIGPLVVAGVACSDEDRLRKLGVKDSKKLSQGRREELAEEIRKICRTEVLKVSPENLDERMAAKTINEILKECYAEIILRLKPEIAYVDSPDVIPERLSRELEEITGLRVVAEHKADEKYPLVAAASIIAKVEREREIERLKEKFGDFGSGYASDPRTREVLKEWIASGRIPSCVRMRWKTVSNLRQKTLDDF

SEQ ID NO: 76 - protein, 233 aa, Thermococcus celer
LKLAGIDEAGRGPVIGPMVIAAVVLDEKNVPKLRDLGVRDSKKLTPKRRERLFNDIIKLLDDYVILELWPEEIDSRGGTLNELEVERFVEALNSLKVKPDVVYIDAADVKEGRFGEEIKERLNFEAKIVSEHRADDKFLPVSSASILAKVTRDRAIEKLKEKYGEIGSGYPSDPRTREFLENYYRQHGEFPPVVRRSWKTLRKIEEKLRKEAGSKNPENSKEKGQTSLDVFLR

SEQ ID NO: 77 - protein, 224 aa, Thermococcus litoralis
MKLGGIDEAGRGPVIGPLVIAAVVVDESRMQELEALGVKDSKKLTPKRREELFEEIVQIVDDHVIIQLSPEEIDGRDGTMNELEIENFAKALNSLKVKPDVLYIDAADVKEKRFGDIIGERLSFSPKIIAEHKADSKYIPVAAASILAKVTRDRAIEKLKELYGEIGSGYPSDPNTRRFLEEYYKAHGEFPPIVRKSWKTLRKIEEKLKAKKTQPTILDFLKKP

SEQ ID NO: 78 - protein, 174 aa, Haemophilus influenzae
MFNLSLSIKIPAILHNNLFVMQKQIEIFTDGSCLGNPGAGGIGAVLRYKQHEKMLSKGYFKTTNNRMELRAVIEALNTLKEPCLITLYSDSQYMKNGITKWIFNWKKNNWKASSGKPVKNQDLWIALDESIQRHKINWQWVKGHAGHRENEICDELAKKGAENPTLEDMGYFEE

SEQ ID NO: 79 - protein, 166 aa, Thermus thermophilus; identical to SEQ ID NO: 65

SEQ ID NO: 80 - protein, 161 aa, Thermus aquaticus
MSLPLKRVDLFTDGACLGNPGPGGWAALLRYGSQEKLLSGGEPCTTNNRMELRAALEGLLALREPCQVHLHTDSQYLKRAFAEGWVERWQRNGWRTAEGKPVKNQDLWQALLKAMEGHEVAFHFVEGHSGHPENERVDREARRQAKAQPQVPCPPKEATLF

SEQ ID NO: 81 - protein, 146 aa, Salmonella enterica
MLKQVEIFTDGSCLGNPGPGGYGAILRYRGHEKTFSEGYTLTTNNRMELMAAIVALEALKEHCEVTLSTDSQYVRQGITQWIHNWKKRGWKTAEKKPVKNVDLWKRLDAALGQHQIKWVWVKGHAGHPENERCDELARAAAMNPTQ

SEQ ID NO: 82 - protein, 146 aa, Agrobacterium tumefaciens
MKHVDIFTDGACSGNPGPGGWGAVLRYGETEKELSGGEADTTNNRMELLAAISALNALKSPCEVDLYTDSAYVKDGITKWIFGWKKKGWKTADNKPVKNVELWQALEAAQERHKVTLHWVKGHAGHPENERADELARKGMEPFKRR

(19) United States
(12) Patent Application Publication
(10) Pub. No.: US 2013/0209987 A1
(43) Pub. Date: Aug. 15, 2013

(54) OLIGONUCLEOTIDE SETS FOR DETECTION OF HUMAN PAPILLOMAVIRUS

(71) Applicant: Jun Li, Beltsville, MD (US)

(72) Inventor: Jun Li, Beltsville, MD (US)

(73) Assignee: SAMSUNG TECHWIN CO., LTD., Changwon-city (KR)

(21) Appl. No.: 13/370,738

(22) Filed: Feb. 10, 2012

(51) Int. Cl. C12Q 1/70; C07H 21/04

(52) U.S. Cl. 435/5; 536/24.33

(57) ABSTRACT

Disclosed are methods and kits for detecting high risk HPV genotypes 16, 18, 31, 33, 35, 39, 45, 51, 52, 56, 58, 59, 66 and 68, that are known to cause abnormal cell growth and cancer. The disclosed methods and kits allow a rapid and quantitative real-time PCR detection of all high risk strains of HPV in a single PCR reaction. The procedure promises to facilitate the rapid high throughput detection of high risk strains of HPV in a cost effective and reliable manner.

### FIELD

The disclosure relates to methods and a kit of reagents for the real-time PCR detection of high risk strains of Human Papillomavirus (HPV).

### BACKGROUND

The Human Papillomavirus (HPV) is the agent responsible for the most prevalent sexually transmitted infection (STI) in the United States and the principal factor associated with the development of cervical cancer. HPV is a non-enveloped virus of icoshedral symmetry with 72 capsomeres that surround a genome containing double-stranded circular DNA with approximately 8000 base pairs. Highly species-specific, humans are the only known reservoir for HPV. More than 150 HPV serotypes have been identified, and the genomes of more than 80 have been completely sequenced.

Whereas most HPV infections give rise to benign tissue growth such as warts or papillomas, a group of approximately 40 sexually transmitted HPV serotypes are termed ‘high-risk’ because they play a fundamental role in the etiology of cervical cancer as well as anal, vaginal, vulvar, penile, oropharyngeal, and squamous cell skin cancers.

The number of patients identified with HPV disease has increased markedly during the past 20 years. For instance, the occurrence of anogenital warts, or condylomata acuminata, a common manifestation of HPV infection, increased 5-fold from 1966-1986 amounting to an estimated 500,000 to 1 million new cases annually.

In the United States alone, 2.5 million women are estimated to have an annual cytological diagnosis of a low-grade cervical cancer precursor. The incidence of overt cervical cancer has however decreased dramatically thanks in part to a greater awareness of the disease and the widespread implementation of the Papanicolaou test (Pap Test, or Pap smear) as a screening tool. Nevertheless, from 1990-2001, the number of estimated new invasive cervical cancers remained relatively constant at about 13,500 annually, in part because current screening assays, such as the PAP smear, are prone to false negative results that contribute to misdiagnosis.

Hence, there is an on-going need for user-friendly, accurate kits for the more reliable detection of high risk HPV infections.

### SUMMARY

High risk HPV include genotypes 16, 18, 31, 33, 35, 39, 45, 51, 52, 56, 58, 59, 66 and 68, that are known to cause abnormal cell growth and cancer. The significant amount of sequence heterogeneity found between the genomes of different high risk HPV genotypes has hampered the development of a convenient and sensitive PCR assay that tests for the presence of all high risk HPV genotypes. Methods and kits disclosed herein allows a rapid and quantitative real-time PCR detection of all high risk strains of HPV in a single PCR reaction. The procedure promises to facilitate the rapid high throughput detection of HPV in a cost effective and reliable manner.

In one embodiment, it is disclosed a population of Human Papillomavirus (HPV)-specific oligonucleotides each having a nucleotide sequence that aligns with any one of the HPV nucleotide sequences of SEQ ID NOs: 31-55, wherein each oligonucleotide within the population comprises at least 10 consecutive nucleotides selected from the nucleotide sequence of GGTAGATACTACHMGYAGYAC (SEQ ID NO: 56), wherein H is A or C or T/U, Y is C or T/U and M is A or C, and wherein the oligonucleotides are less than about 35 nucleotides in length.

In another embodiment, it is disclosed a population of Human Papillomavirus (HPV)-specific oligonucleotides each having a nucleotide sequence that aligns with any one of the HPV nucleotide sequences of SEQ ID NOs: 31-55, wherein each oligonucleotide within the population comprises at least 5 consecutive nucleotides selected from the nucleotide sequence of ATACTACHMGYAGYAC (SEQ ID NO: 70), wherein H is A or C or T/U, Y is C or T/U and M is A or C, and wherein the oligonucleotides are less than about 35 nucleotides in length.

Each oligonucleotide within the population may include the sequence TACHMGYAGYAC (SEQ ID NO: 57), wherein H is A or C or T/U, Y is C or T/U and M is A or C.

In another embodiment, a population of Human Papillomavirus (HPV)-specific oligonucleotides each having a nucleotide sequence that aligns with the complementary nucleotide sequence of any one of the HPV nucleotide sequences of SEQ ID NOs: 31-55 is disclosed, wherein each oligonucleotide within the population comprises at least 10 consecutive nucleotides selected from the nucleotide sequence of TGTAAATCATAYT (SEQ ID NO: 58), wherein Y is C or T/U, and wherein the oligonucleotides are less than about 35 nucleotides in length.

In another embodiment, a population of Human Papillomavirus (HPV)-specific oligonucleotides each having a nucleotide sequence that aligns with the complementary nucleotide sequence of any one of the HPV nucleotide sequences of SEQ ID NOs: 31-55 is disclosed, wherein each oligonucleotide within the population comprises at least 5 consecutive nucleotides selected from the nucleotide sequence of ATCATAYT,

wherein Y is C or T/U, and wherein the oligonucleotides are less than about 35 nucleotides in length.

Each oligonucleotide within the population can include the sequence AATCAATCATAYT (SEQ ID NO: 59), wherein H is A or C or T/U, Y is C or T/U and M is A or C.

In one embodiment, it is disclosed a kit for the simultaneous real-time PCR detection of high risk Human Papillomavirus (HPV) genotypes comprising a forward amplification primer having the nucleotide sequence of SEQ ID NO: 1.

In one embodiment, it is disclosed a kit for the real-time PCR detection of high risk Human Papillomavirus (HPV) genotypes comprising a reverse amplification primer having the nucleotide sequence of SEQ ID NO: 16.

The kit may include a DNA and/or RNA dependent DNA polymerase activity.

In another embodiment, it is disclosed a method for the real-time detection of high risk Human Papillomavirus (HPV) genotypes in a sample, comprising the steps of:

providing a sample to be tested for the presence of high risk HPV genotype DNA;

providing a forward amplification primer having the nucleotide sequence of SEQ ID NO: 1 and a reverse amplification primer having the nucleotide sequence of SEQ ID NO: 16, wherein the forward and reverse primers simultaneously anneal to target HPV DNA sequences;

amplifying a PCR fragment between the forward and reverse amplification primers in the presence of an amplification buffer comprising an amplifying polymerase activity and a fluorescent dye, and

detecting a real-time increase in the emission of a fluorescent signal,

wherein the increase in the fluorescent signal indicates the presence of one or more high risk HPV genotypes in the sample.

In another embodiment, it is disclosed a method for the real-time PCR detection of high risk HPV in a sample, comprising the steps of:

providing a sample to be tested for the presence of high risk HPV genotype RNA;

providing a forward amplification primer having the nucleotide sequence of SEQ ID NO: 1 and a reverse amplification primer having the nucleotide sequence of SEQ ID NO: 16, wherein the forward and reverse primers simultaneously anneal to target HPV nucleic acid sequences;

reverse transcribing high risk HPV RNAs in the presence of a reverse transcriptase buffer comprising reverse transcriptase activity and the reverse amplification primer to produce a target a high risk HPV cDNA sequence;

amplifying a PCR fragment between the forward and reverse amplification primers in the presence of the target HPV cDNA sequence and an amplification buffer comprising an amplifying polymerase activity and a fluorescent dye, and

detecting a real-time increase in the emission of a fluorescent signal,

wherein the increase in the fluorescent signal indicates the presence of one or more high risk HPV genotypes in the sample.

The high risk HPV genotypes can include HPV genotypes 16, 18, 31, 33, 35, 39, 45, 51, 52, 56, 58, 59, 66 and 68. The HPV target DNA sequences can include the nucleotide sequences of SEQ ID NOs: 31-55.

The increase in the fluorescent signal can detect the presence of about 100 copies of HPV DNA from genotypes 16, 18, 31, 33, 35, 39, 45, 52, 58, 59, 66 and 68 and about 1,000 copies of HPV DNA from genotype 51.

The amplifying polymerase activity can be the activity of a thermostable DNA polymerase. The fluorescent dye can be SYBR™ Green I. The PCR fragment can be linked to a solid support and the nucleic acid within the sample can be pre-treated with uracil-N-glycosylase.

The above described embodiments have many advantages, including the ability to detect HPV nucleic acid sequences in real-time. The detection method is fast, accurate and suitable for high throughput applications. Convenient and user-friendly diagnostic kits are also described for the rapid and reliable detection of high risk HPV strains.

### BRIEF DESCRIPTION OF THE DRAWINGS

The skilled artisan will understand that the drawings, described below, are for illustration purposes only. The figures are not intended to limit the scope of the teachings in any way.

FIG. 1 depicts a sequence alignment of a 140 bp conserved region within the L1 open reading frame of high risk HPV genotypes 16, 18, 31, 33, 35, 39, 45, 51, 52, 56, 58, 59, 66 and 68.

FIG. 2 is a schematic representation of CataCleave™ probe technology.

FIG. 3 is a schematic representation of real-time CataCleave™ probe detection of PCR amplification products.

FIG. 4 shows amplification curves obtained by real-time polymerase chain reaction (PCR) of high risk HPV genotypes (FIG. 4A: HPV genotype 16, FIG. 4B: HPV genotype 18, FIG. 4C: HPV genotype 31, FIG. 4D: HPV genotype 33, FIG. 4E: HPV genotype 35, FIG. 4F: HPV genotype 39, FIG. 4G: HPV genotype 45, FIG. 4H: HPV genotype 51, FIG. 4I: HPV genotype 52,

FIG. 4J: HPV genotype 56, FIG. 4K: HPV genotype 58, FIG. 4L: HPV genotype 59, FIG. 4M: HPV genotype 66 and FIG. 4N: HPV genotype 68).

FIG. 5 depicts gel electrophoresis of PCR amplification of high risk HPV genotypes 16, 18, 31, 33, 35, 39, 45, 51, 52, 56, 58, 59, 66 and 68.

FIG. 6 depicts a sequence alignment between Pyrococcus furiosis, Pyrococcus horikoshi, Thermococcus kodakarensis, Archeoglobus profundus, Archeoglobus fulgidis, Thermococcus celer and Thermococcus litoralis RNase HII polypeptide sequences.

FIG. 7 depicts sequence alignment of Haemophilus influenzae, Thermus thermophilis, Thermus acquaticus, Salmonella enterica and Agrobacterium tumefaciens RNase HI polypeptide sequences.

### DETAILED DESCRIPTION

The practice of the invention employs, unless otherwise indicated, conventional molecular biological techniques within the skill of the art. Such techniques are well known to the skilled worker, and are explained fully in the literature. See, e.g., Ausubel, et al., ed., Current Protocols in Molecular Biology, John Wiley & Sons, Inc., NY, N.Y. (1987-2008), including all supplements; Sambrook, et al., Molecular Cloning: A Laboratory Manual, 2nd Edition, Cold Spring Harbor, N.Y. (1989).

Unless defined otherwise, all technical and scientific terms used herein have the same meaning as is commonly understood by one of skill in the art. The specification also provides definitions of terms to help interpret the disclosure and claims of this application. In the event a definition is not consistent with definitions elsewhere, the definitions set forth in this application will control.

As used herein, the term “base” refers to any nitrogen-containing heterocyclic moiety capable of forming Watson-Crick type hydrogen bonds in pairing with a complementary base or base analog. A large number of natural and synthetic (non-natural, or unnatural) bases, base analogs and base derivatives are known. Examples of bases include purines, pyrimidines, and modified forms thereof. The naturally occurring bases include, but are not limited to, adenine (A), guanine (G), cytosine (C), uracil (U) and thymine (T). As used herein, it is not intended that the invention be limited to naturally occurring bases, as a large number of unnatural (non-naturally occurring) bases and their respective unnatural nucleotides that find use with the invention are known to one of skill in the art.

The term “nucleoside” refers to a compound consisting of a base linked to the C-1′ carbon of a sugar, for example, ribose or deoxyribose.

The term “nucleotide” refers to a phosphate ester of a nucleoside, as a monomer unit or within a polynucleotide. The term “nucleotide,” as used herein, refers to a compound comprising a nucleotide base linked to the C-1′ carbon of a sugar, such as ribose, arabinose, xylose, and pyranose, and sugar analogs thereof. The term nucleotide also encompasses nucleotide analogs. The sugar may be substituted or unsubstituted. Substituted ribose sugars include, but are not limited to, those riboses in which one or more of the carbon atoms, for example the 2′-carbon atom, is substituted with one or more of the same or different C1, F, —R, —OR, —NR₂ or halogen groups, where each R is independently H, C1-C6 alkyl or C5-C14 aryl. Exemplary riboses include, but are not limited to, 2′-(C1-C6)alkoxyribose, 2′-(C5-C14)aryloxyribose, 2′,3′-didehydroribose, 2′-deoxy-3′-haloribose, 2′-deoxy-3′-fluororibose, 2′-deoxy-3′-chlororibose, 2′-deoxy-3′-aminoribose, 2′-deoxy-3′-(C1-C6)alkylribose, 2′-deoxy-3′-(C1-C6)alkoxyribose and 2′-deoxy-3′-(C5-C14)aryloxyribose, ribose, 2′-deoxyribose, 2′,3′-dideoxyribose, 2′-haloribose, 2′-fluororibose, 2′-chlororibose, and 2′-alkylribose, e.g., 2′-O-methyl, 4′-α-anomeric nucleotides, 1′-α-anomeric nucleotides, 2′-4′- and 3′-4′-linked and other “locked” or “LNA”, bicyclic sugar modifications (see, e.g., PCT published application nos. WO 98/22489, WO 98/39352, and WO 99/14226; and U.S. Pat. Nos. 6,268,490 and 6,794,499). Further synthetic nucleotides having modified base moieties and/or modified sugar moieties, e.g., as described by Scheit: Nucleotide Analogs (John Wiley New York, 1980); Uhlman and Peyman, 1990, Chemical Reviews 90:543-584.

The terms “polynucleotide,” “nucleic acid,” “oligonucleotide,” “oligomer,” “oligo,” primer or equivalent terms, as used herein refer to a polymeric arrangement of monomers that can be corresponded to a sequence of nucleotide bases, such as deoxyribonucleic acid (DNA), ribonucleic acid (RNA), and, where appropriate, phosphothioate containing nucleic acids, locked nucleic acid (LNA), peptide nucleic acid (PNA), or other derivative nucleic acid molecules and combinations thereof.

Nucleic acids include, but are not limited to, high risk HPV synthetic DNA, plasmid DNA, genomic DNA, cDNA, mRNA or total comprising HPV nucleic acid sequences.

Polynucleotides are polymers of nucleotides comprising two or more nucleotides. Polynucleotides may be double-stranded nucleic acids, including annealed oligonucleotides wherein the second strand is an oligonucleotide with the reverse complement sequence of the first oligonucleotide, single-stranded nucleic acid polymers comprising deoxythymidine, single-stranded RNAs, double stranded RNAs or RNA/DNA heteroduplexes or single-stranded nucleic acid polymers comprising double stranded regions e.g. DNA hairpin loops and/or RNA hairpin loops and/or DNA/RNA hairpin loops.

As used herein, an “oligonucleotide” refers to a short polynucleotide. In certain embodiments, an oligonucleotide may be about 10, about 20, about 30, about 40, about 50 or more 60 nucleotides in length. In other embodiments, an oligonucleotide is less than about 500 nucleotides, less than about 250 nucleotides, less than about 200 nucleotides, less than about 150 nucleotide or less than 100 nucleotides.

Oligonucleotides or polynucleotides may be modified or may comprise modified bases or modified or non-naturally occurring sugar residues. Several reviews on modified oligonucleotides, including conjugates have been published; see for example, Verma and Eckstein Annu. Rev. Biochem. (1998) 67:99-134, Uhlmann and Peyman, Chemical Reviews, Vol. 90, pgs. 543-584 (1990), and Goodchild, Bioconjugate Chemistry, Vol. 1, pgs 165-187 (1990), Cobb Org Biomol Chem. (2007) 5(20):3260-75, Lyer et al. Curr Opin Mol. Ther. (1999) 1(3):344-58), U.S. Pat. Nos. 6,172,208, 5,872,244 and published U.S. Patent Application No. 2007/0281308.

In certain embodiments, oligonucleotides may comprise about 1, about 2, about 3, about 4, about 5 or more degenerate nucleotides. Degenerate nucleotides may be complementary, non-complementary, or partially non-complementary. Complementarity between nucleotides refers to the ability to form a Watson-Crick base pair through specific hydrogen bonds (e.g., A and T base pair via two hydrogen bonds; and C and G are base pair via three hydrogen bonds).

TABLE A

| Symbol | Origin of Symbol | Meaning* | Complimentary |
|---|---|---|---|
| K | keto | G or T/U | Non-complementary |
| R | purine | A or G | Non-complementary |
| Y | pyrimidine | C or T/U | Non-complementary |
| S | Strong interaction | C or G | Complementary |
| W | Weak interaction | A or T/U | Complementary |
| B | not A | C or G or T/U | Partially |
| | | | non-complementary |
| D | not C | A or G or T/U | Partially |
| | | | non-complementary |
| H | not G | A or C or T/U | Partially |
| | | | non-complementary |
| V | not T/U | A or C or G | Partially |
| | | | non-complementary |
| N | any | A or C or G or T/U | Complementary |

*A = adenosine, C = cytidine, G = guanosine, T = thymidine, U = uridine

A “primer dimer” is a potential by-product in PCR, that consists of primer molecules that have partially hybridized to each other because of strings of complementary bases in the primers. As a result, the DNA polymerase amplifies the primer dimer, leading to competition for PCR reagents, thus potentially inhibiting amplification of the DNA sequence targeted for PCR amplification.

The term “template nucleic acid” refers to a plurality of nucleic acid molecules used as the starting material or template for amplification in a PCR reaction or reverse transcriptase-PCR reaction. Template nucleic acid sequences may include both naturally occurring and synthetic molecules. Exemplary template nucleic acid sequences include, but are not limited to, genomic HPV DNA or total RNA comprising HPV RNA sequences.

A “target DNA” or “target RNA” or “target nucleic acid,” or “target nucleic acid sequence” refers to a region of a template nucleic acid that is to be analyzed.

As used herein, the term “amplification primer” or “PCR primer” or “primer” refers to an enzymatically extendable oligonucleotide that comprises a defined sequence that is designed to hybridize in an antiparallel manner with a complementary, primer-specific portion of a target nucleic acid sequence. Thus, the primer, which is generally in molar excess relative to its target polynucleotide sequence, primes template-dependent enzymatic DNA synthesis and amplification of the target sequence. A primer nucleic acid does not need to have 100% complementarity with its template subsequence for primer elongation to occur. Primers can be “substantially complementary” to a target template nucleic acid sequence provided the complementarity is sufficient for hybridization and polymerase elongation to occur and provided the penultimate base at the 3′ end of the primer is able to base pair with the template nucleic acid. A PCR primer is preferably, but not necessarily, synthetic, and will generally be approximately about 10 to about 100 nucleotides in length.

Oligonucleotides may be synthesized and prepared by any suitable method (such as chemical synthesis), which is known in the art. A number of computer programs (e.g., Primer-Express) are readily available to design optimal primer sets. One of the skilled artisans would therefore easily optimize and identify primers flanking a target nucleic acid sequence of interest. For example, synthesized primers can be between 20 and 26 base pairs in length with a melting point (TM) of around 55 degrees. Hence, it will be apparent to one of skill in the art that the primers and probes based on the nucleic acid information provided (or publicly available with accession numbers) can be prepared accordingly.

The terms “annealing” and “hybridization” are used interchangeably and mean the base-pairing interaction of one nucleic acid with another nucleic acid that results in formation of a duplex, triplex, or other higher-ordered structure. In certain embodiments, the primary interaction is base-specific, e.g., A/T and G/C, by Watson/Crick and Hoogsteen-type hydrogen bonding. In certain embodiments, base-stacking and hydrophobic interactions may also contribute to duplex stability. “Substantially complimentary” refers to two nucleic acid strands that are sufficiently complimentary in sequence to anneal and form a stable duplex.

### Design of Primer Sequences for the Detection of High Risk HPV Gentotypes

To select high risk HPV target nucleic acid sequences for real-time PCR detection, the complete genome sequences of different HPV virus lineages are first aligned and examined for regions of homology. Matrix comparison of the sequenced HPV types 1a, 6b, 16 and 18 previously revealed that the most conserved regions are localized within the E1 and L1 ORFs (Gini & Danos (1986). Papillomavirus genomes. From sequence data to biological properties. Trends in Genetics 2, 227-232). Consequently, these homology regions were screened for potential primer pairs that could be used for the detection of all high risk HPV sequences.

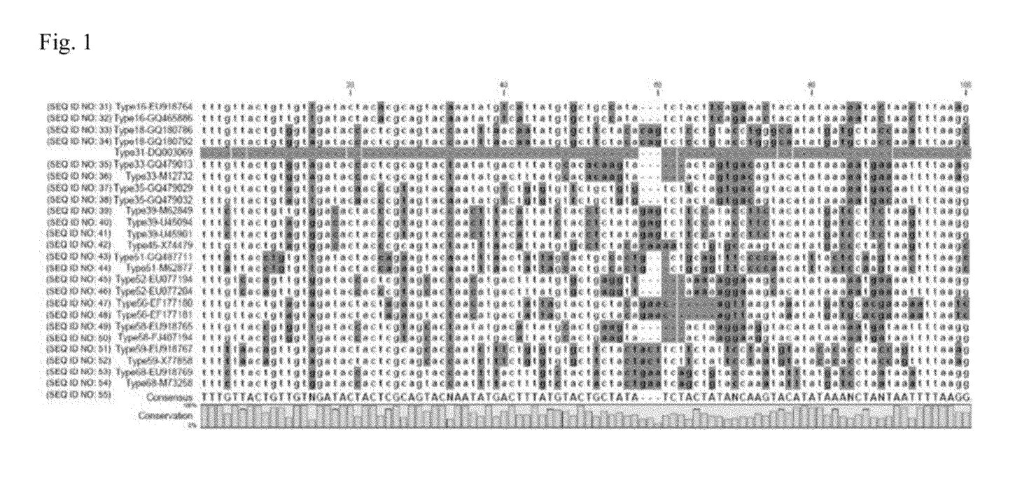

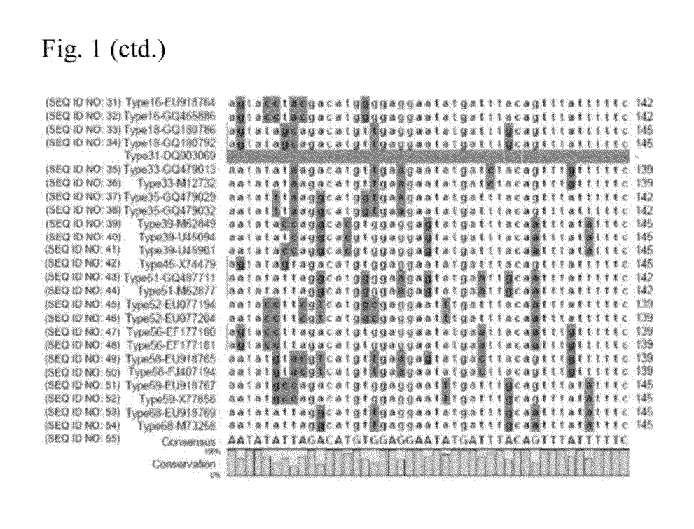

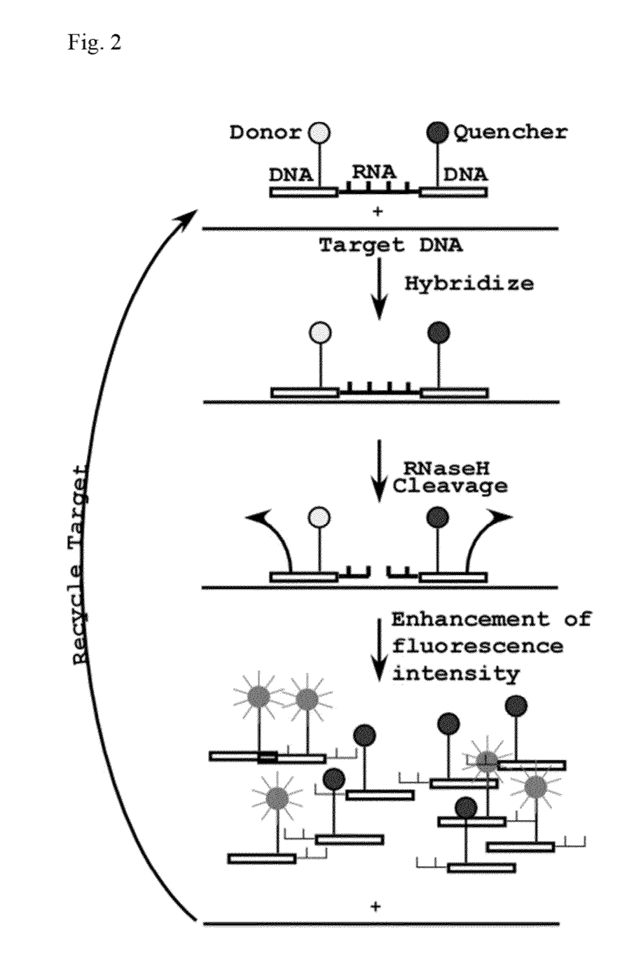

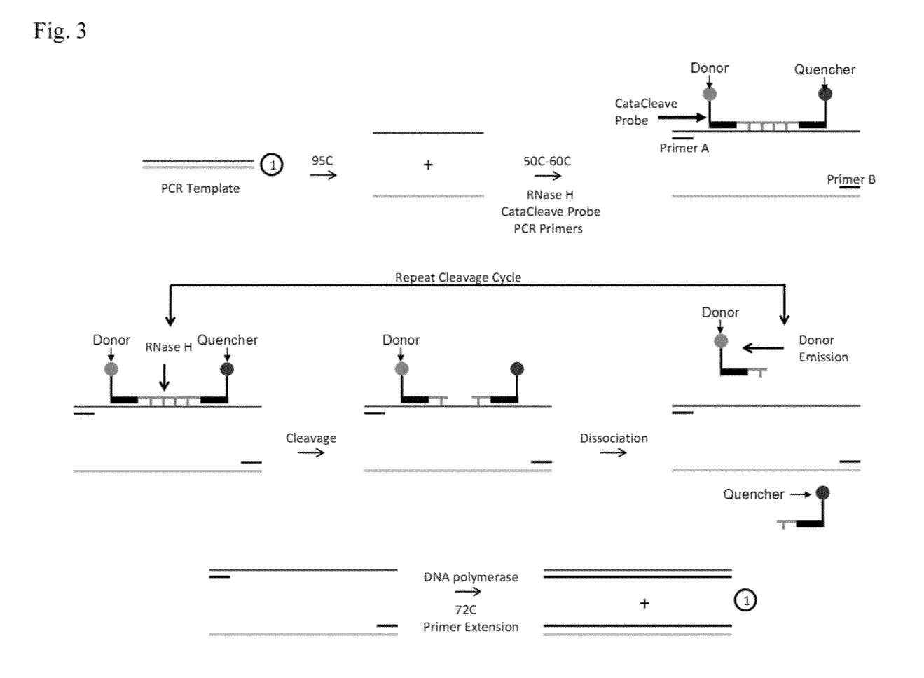

FIG. 1 depicts a sequence alignment within a 140 bp conserved region within the L1 open reading frame of high risk HPV genotypes 16, 18, 31, 33, 35, 39, 45, 51, 52, 56, 58, 59, 66 and 68.

Sequence alignment in the forward primer region is shown below:

| Name | Sequence | Sequence Identifier |
|---|---|---|
| HPV_HR_F10D | TTT GTT ACT GTG GTA GAT ACT ACH MGY AGY AC | SEQ ID NO: 1 |
| HPV Type 16 | TTT GTT ACT GTT GTT GAT ACT ACA CGC AGT AC | SEQ ID NO: 2 |
| HPV Type 18 | TTT GTT ACT GTG GTA GAT ACC ACT CGC AGT AC | SEQ ID NO: 3 |
| HPV Type 31 | TTT GTT ACT GTG GTA GAT ACC ACA CGT AGT AC | SEQ ID NO: 4 |
| HPV Type 33 | TTT GTT ACT GTG GTA GAT ACC ACT CGC AGT AC | SEQ ID NO: 5 |
| HPV Type 35 | TTT GTT ACT GTA GTT GAT ACA ACC CGT AGT AC | SEQ ID NO: 6 |
| HPV Type 39 | TTT CTT ACT GTT GTG GAC ACT ACC CGT AGT AC | SEQ ID NO: 7 |
| HPV Type 45 | TTT GTT ACT GTA GTG GAC ACT ACC CGC AGT AC | SEQ ID NO: 8 |
| HPV Type 51 | TTT ATT ACC TGT GTT GAT ACT ACC AGA AGT AC | SEQ ID NO: 9 |
| HPV Type 52 | TTT GTC ACA GTT GTG GAT ACC ACT CGT AGC AC | SEQ ID NO: 10 |
| HPV Type 56 | TTT GTT ACT GTA GTA GAT ACT ACT AGA AGT AC | SEQ ID NO: 11 |
| HPV Type 58 | TTT GTT ACC GTG GTT GAT ACC ACT CGT AGC AC | SEQ ID NO: 12 |
| HPV Type 59 | TTT TTA ACA GTT GTA GAT ACT ACT CGC AGC AC | SEQ ID NO: 13 |
| HPV Type 66 | TTT GTT ACT GTT GTG GAT ACT ACC AGA AGT AC | SEQ ID NO: 14 |
| HPV Type 68 | TTT CTT ACT GTT GTG GAT ACC ACT CGC AGT AC | SEQ ID NO: 15 |

Sequence alignment in the reverse primer region is shown below:

| | | |
|---|---|---|
| HPV_HR_R4 | GAA AAA TAA ACT GTA | SEQ ID NO: 16 |
| | AAT CAT AYT | |
| HPV Type 16 | GAA AAA TAA ACT GTA | SEQ ID NO: 17 |
| | AAT CAT ATT | |
| HPV Type 18 | GAA AAA TAA ACT GCA | SEQ ID NO: 18 |
| | AAT CAT ATT | |
| HPV Type 31 | GAA ATA TAA ATT GTA | SEQ ID NO: 19 |
| | AAT CAA ATT | |
| HPV Type 33 | GAA AAA CAA ACT GTA | SEQ ID NO: 20 |
| | GAT CAT ATT | |
| HPV Type 35 | GAA AAA TAA ACT GTA | SEQ ID NO: 21 |
| | AAT CAT ATT | |
| HPV Type 39 | GAA ATA TAA ATT GTA | SEQ ID NO: 22 |
| | AAT CAT ACT | |
| HPV Type 45 | GAA AAA TAA ACT GTA | SEQ ID NO: 23 |
| | AAT CAT ATT | |
| HPV Type 51 | GAA AAA TAA ATT GCA | SEQ ID NO: 24 |
| | ATT CAT ACT | |
| HPV Type 52 | GAA AAA TAA ATT GTA | SEQ ID NO: 25 |
| | AAT CAA ATT | |
| HPV Type 56 | GAA AAA CAA ATT GTA | SEQ ID NO: 26 |
| | ATT CAT ATT | |
| HPV Type 58 | GAA AAA CAA ACT GTA | SEQ ID NO: 27 |
| | AGT CAT ACT | |
| HPV Type 59 | GAA ATA TAA ACT GCA | SEQ ID NO: 28 |
| | AAT CAA ATT | |
| HPV Type 66 | GAA ACA CAA ACT GTA | SEQ ID NO: 29 |
| | GTT CAT ATT | |
| HPV Type 68 | GAA ATA TAA ATT GCA | SEQ ID NO: 30 |
| | AAT CAT ATT | |

From these sequence alignments, a forward primer, HPV_HR_F10D, 5′-TTTGTTACTGTGGTAGATACTACHMGYAGYAC-3′ (SEQ ID NO: 1), and a reverse primer, HPV_HR_R4,5′-GAAAAATAAACTGTAAATCATAYT-3′ (SEQ ID NO: 16), where H is A or C or T/U, Y is C or T/U and M is A or C, were designed to optimize detection sensitivity by incorporating degenerate nucleotides at positions of sequence heterogeneity between the different genotypes and selecting for high stringency primer annealing temperatures.

In certain embodiments, the oligonucleotides of SEQ ID NOs: 1-30 can also be used as HPV hybridization probes.

### Nucleic Acid Template Preparation—DNA Template

High risk HPV nucleic acid templates can be derived from human cervical tissue biopsy samples or microorganisms comprising HPV recombinant nucleic acids such as plasmid, phage or viral vectors.

Procedures for the extraction and purification of nucleic acids from samples are well known in the art (as described in Sambrook J et. al. Molecular Cloning, Cold Spring harbor Laboratory Press (1989), Ausubel et al. Short Protocols in Molecular Biology, 5th Ed. (2002) John Wiley & Sons, Inc. New York).

In addition, several commercial kits are available for the isolation of nucleic acids. Exemplary kits include, but are not limited to, Puregene DNA isolation kit (PG) (Gentra Systems, Inc., Minneapolis, Minn.), Generation Capture Column kit (GCC) (Gentra Systems, Inc.), MasterPure DNA purification kit (MP) (Epicentre Technologies, Madison, Wis.), Isoquick nucleic acid extraction kit (IQ) (Epoch Pharmaceuticals, Bothell, Wash.), NucliSens isolation kit (NS) (Organon Teknika Corp., Durham, N.C.), QIAamp DNA Blood Mini Kit (Qiagen; Cat. No. 51104), MagNA Pure Compact Nucleic Acid Isolation Kit (Roche Applied Sciences; Cat. No. 03730964001), Stabilized Blood-to-CT™ Nucleic Acid Preparation Kit for qPCR (Invitrogen, Cat. No. 4449080) and GF-1 Viral Nucleic Acid Extraction Kit (GeneOn, Cat. No. RD05).

### Nucleic Acid Template Preparation—RNA Template

In some embodiments, the sample is a purified RNA template (e.g., HPV viral mRNA, total RNA, and mixtures thereof). In other embodiments, the sample may include cells collected from a PAP smear or a lysate of cultured cells but is not limited thereto. Cells can be frozen on dry ice and stored at −70° C. prior to RNA isolation.

Procedures for the extraction and purification of RNA from samples are well known in the art. For example, total RNA can be isolated from cells using the TRIzol™ reagent (Invitrogen) extraction method. RNA quantity and quality is then determined using, for example, a Nanodrop™ spectrophotometer and an Agilent 2100 bioanalyzer (see also Peirson S N, Butler J N (2007). “RNA extraction from mammalian tissues” Methods Mol. Biol. 362: 315-27, Bird IM (2005) “Extraction of RNA from cells and tissue” Methods Mol. Med. 108: 139-48). In addition, several commercial kits are available for the isolation of RNA. Exemplary kits include, but are not limited to, RNeasy and QIAamp Viral RNA Kit (Qiagen, Valencia, Calif.) and MagMAX™ Viral RNA Isolation Kits (Ambion).

In other embodiments, HPV RNA sequences can be obtained by T7 RNA transcription of HPV L1 gene sequences (SEQ ID NOs: 31-55). An exemplary commercial kit for T7 in vitro transcription is Ambion's MEGAscript® Kit (Catalog No. 1330).

### PCR Amplification of Target Nucleic Acid Sequences

HPV nucleic acid amplification can be accomplished by a variety of methods, including, but not limited to, the polymerase chain reaction (PCR), nucleic acid sequence based amplification (NASBA), ligase chain reaction (LCR), strand displacement amplification (SDA) reaction, transcription mediated amplification (TMA) reaction, and rolling circle amplification (RCA). The polymerase chain reaction (PCR) is the method most commonly used to amplify specific target DNA sequences.

“Polymerase chain reaction,” or “PCR,” generally refers to a method for amplification of a desired nucleotide sequence in vitro. Generally, the PCR process consists of introducing a molar excess of two or more extendable oligonucleotide primers to a reaction mixture comprising a sample having the desired target sequence(s), where the primers are substantially complementary to opposite strands of the double stranded target sequence. The reaction mixture is subjected to a program of thermal cycling in the presence of an amplifying nucleic acid polymerase, resulting in the amplification of the desired target sequence flanked by the sequence-specific primers.

As used herein, an “amplifying polymerase activity” refers to an enzymatic activity that catalyzes the polymerization of deoxyribonucleotides or ribonucleotides. Generally, the enzyme will initiate synthesis at the 3′ end of the primer annealed to a target nucleic acid template sequence, and will proceed toward the 5′ end of the template strand.

The amplifying nucleic acid polymerase can have one or more of the activities of a DNA-dependent DNA polymerase, a DNA-dependent RNA polymerase, a RNA-dependent DNA polymerase or a RNA dependent RNA polymerase.

A “DNA-dependent DNA polymerase activity” refers to the activity of a DNA polymerase enzyme that uses deoxyribonucleic acid (DNA) as a template for the synthesis of a complementary and anti-parallel DNA strand.

A “DNA-dependent RNA polymerase activity” refers to the activity of an RNA polymerase enzyme that uses deoxyribonucleic acid (DNA) as a template for the synthesis of an RNA strand in a process called “transcription.” (for example, Thermo T7 RNA polymerase, commercially available from Toyobo Life Science Department, Catalogue No. TRL-201)

A “RNA-dependent DNA polymerase activity” refers to the activity of a DNA polymerase enzyme that uses ribonucleic acid (RNA) as a template for the synthesis of a complementary and anti-parallel DNA strand in a process called “reverse transcription.”

A “RNA-dependent RNA polymerase activity” refers to the activity of a RNA polymerase enzyme that uses ribonucleic acid (RNA) as a template for the synthesis of a complementary RNA strand (for example, Thermus thermophilus RNA polymerase, commercially available from Cambio, Catalogue No. T90250).

### DNA Polymerase PCR Amplification

In certain embodiments, the nucleic acid polymerase is a thermostable polymerase that may have more than one of the above-specified catalytic activities.

As used herein, the term “thermostable,” as applied to an enzyme, refers to an enzyme that retains its biological activity at elevated temperatures (e.g., at 55° C. or higher), or retains its biological activity following repeated cycles of heating and cooling.

Non-limiting examples of thermostable amplifying polymerases having “DNA-dependent DNA polymerase activity” include, but are not limited to, polymerases isolated from the thermophilic bacteria Thermus aquaticus (Taq polymerase), Thermus thermophilus (Tth polymerase), Thermococcus litoralis (Tli or VENT™ polymerase), Pyrococcus furiosus (Pfu or DEEPVENT™. polymerase), Pyrococcus woosii (Pwo polymerase) and other Pyrococcus species, Bacillus stearothermophilus (Bst polymerase), Sulfolobus acidocaldarius (Sac polymerase), Thermoplasma acidophilum (Tac polymerase), Thermus rubber (Tru polymerase), Thermus brockianus (DYNAZYME™ polymerase) i (Tne polymerase), Thermotoga maritime (Tma) and other species of the Thermotoga genus (Tsp polymerase), and Methanobacterium thermoautotrophicum (Mth polymerase). The PCR reaction may contain more than one thermostable polymerase enzyme with complementary properties leading to more efficient amplification of target sequences. For example, a nucleotide polymerase with high processivity (the ability to copy large nucleotide segments) may be complemented with another nucleotide polymerase with proofreading capabilities (the ability to correct mistakes during elongation of target nucleic acid sequence), thus creating a PCR reaction that can copy a long target sequence with high fidelity. The thermostable polymerase may be used in its wild type form. Alternatively, the polymerase may be modified to contain a fragment of the enzyme or to contain a mutation that provides beneficial properties to facilitate the PCR reaction.

In one embodiment, the thermostable polymerase may be Taq DNA polymerase. Many variants of Taq polymerase with enhanced properties are known and include, but are not limited to, AmpliTaq™, AmpliTaq™, Stoffel fragment, SuperTaq™, SuperTaq™, plus, LA Taq™, LApro Taq™, and EX Taq™. In another embodiment, the thermostable polymerase is the AmpliTaq Stoffel fragment.

The technique of PCR is described in numerous publications, including, PCR: A Practical Approach, M. J. McPherson, et al., IRL Press (1991), PCR Protocols: A Guide to Methods and Applications, by Innis, et al., Academic Press (1990), and PCR Technology: Principals and Applications for DNA Amplification, H. A. Erlich, Stockton Press (1989). PCR is also described in many U.S. Patents, including U.S. Pat. Nos. 4,683,195; 4,683,202; 4,800,159; 4,965,188; 4,889,818; 5,075,216; 5,079,352; 5,104,792; 5,023,171; 5,091,310; and 5,066,584, each of which is herein incorporated by reference.

The term “sample” refers to any substance containing nucleic acid material.

As used herein, the term “PCR fragment” or “reverse transcriptase-PCR fragment” or “amplicon” refers to a polynucleotide molecule (or collectively the plurality of molecules) produced following the amplification of a particular target nucleic acid. A PCR fragment is typically, but not exclusively, a DNA PCR fragment. A PCR fragment can be single-stranded or double-stranded, or in a mixture thereof in any concentration ratio. A PCR fragment or RT-PCT can be about 100 to about 500 nt or more in length.

A “buffer” is a compound added to an amplification reaction which modifies the stability, activity, and/or longevity of one or more components of the amplification reaction by regulating the pH of the amplification reaction. The buffering agents of the invention are compatible with PCR amplification and site-specific RNase H cleavage activity. Certain buffering agents are well known in the art and include, but are not limited to, Tris, Tricine, MOPS (3-(N-morpholino) propanesulfonic acid), and HEPES (4-(2-hydroxyethyl)-1-piperazineethanesulfonic acid). In addition, PCR buffers may generally contain up to about 70 mM KCl and about 1.5 mM or higher MgCl2, to about 50-200 μM each of nucleotides dATP, dCTP, dGTP and dTTP. The buffers of the invention may contain additives to optimize efficient reverse transcriptase-PCR or PCR reaction.

An additive is a compound added to a composition which modifies the stability, activity, and/or longevity of one or more components of the composition. In certain embodiments, the composition is an amplification reaction composition. In certain embodiments, an additive inactivates contaminant enzymes, stabilizes protein folding, and/or decreases aggregation. Exemplary additives that may be included in an amplification reaction include, but are not limited to, formamide, KCl, CaCl₂, Mg(OAc)₂, MgCl₂, NaCl, NH₄OAc, NaI, Na₂CO3, LiCl, Mn(OAc)₂, NMP, trehalose, demethylsulfoxide (“DMSO”), glycerol, ethylene glycol, dithiothreitol (“DTT”), pyrophosphatase (including, but not limited to Thermoplasma acidophilum inorganic pyrophosphatase (“TAP”)), bovine serum albumin (“BSA”), propylene glycol, glycinamide, CHES, Percoll™, aurintricarboxylic acid, Tween 20, Tween 21, Tween 40, Tween 60, Tween 85, Brij 30, NP-40, Triton X-100, CHAPS, CHAPSO, Mackernium, LDAO (N-dodecyl-N,N-dimethylamine-N-oxide), Zwittergent 3-10, Xwittergent 3-14, Xwittergent SB 3-16, Empigen, NDSB-20, T4G32, E. Coli SSB, RecA, nicking endonucleases, 7-deazaG, dUTP, UNG, anionic detergents, cationic detergents, non-ionic detergents, zwittergent, sterol, osmolytes, cations, and any other chemical, protein, or cofactor that may alter the efficiency of amplification. In certain embodiments, two or more additives are included in an amplification reaction. According to the invention, additives may be added to improve selectivity of primer annealing provided the additives do not adversely interfere with the PCR amplification reaction.

### Reverse Transcriptase-PCR Amplification

One of the most widely used techniques to study gene expression exploits first-strand cDNA for mRNA sequence(s) as template for amplification by the PCR.

The term “reverse transcriptase activity” and “reverse transcription” refers to the enzymatic activity of a class of polymerases characterized as RNA-dependent DNA polymerases that can synthesize a DNA strand (i.e., complementary DNA, cDNA) utilizing an RNA strand as a template.

“Reverse transcriptase-PCR” of “RNA PCR” is a PCR reaction that uses RNA template and a reverse transcriptase, or an enzyme having reverse transcriptase activity, to first generate a single stranded DNA molecule prior to the multiple cycles of DNA-dependent DNA polymerase primer elongation. Multiplex PCR refers to PCR reactions that produce more than one amplified product in a single reaction, typically by the inclusion of more than two primers in a single reaction.

Exemplary reverse transcriptases include, but are not limited to, the Moloney murine leukemia virus (M-MLV) RT as described in U.S. Pat. No. 4,943,531, a mutant form of M-MLV-RT lacking RNase H activity as described in U.S. Pat. No. 5,405,776, bovine leukemia virus (BLV) RT, Rous sarcoma virus (RSV) RT, Avian Myeloblastosis Virus (AMV) RT and reverse transcriptases disclosed in U.S. Pat. No. 7,883,871.

The reverse transcriptase-PCR procedure, carried out as either an end-point or real-time assay, involves two separate molecular syntheses: (i) the synthesis of cDNA from an RNA template; and (ii) the replication of the newly synthesized cDNA through PCR amplification. To attempt to address the technical problems often associated with reverse transcriptase-PCR, a number of protocols have been developed taking into account the three basic steps of the procedure: (a) the denaturation of RNA and the hybridization of reverse primer; (b) the synthesis of cDNA; and (c) PCR amplification. In the so called “uncoupled” reverse transcriptase-PCR procedure (e.g., two step reverse transcriptase-PCR), reverse transcription is performed as an independent step using the optimal buffer condition for reverse transcriptase activity. Following cDNA synthesis, the reaction is diluted to decrease MgCl2, and deoxyribonucleoside triphosphate (dNTP) concentrations to conditions optimal for Taq DNA Polymerase activity, and PCR is carried out according to standard conditions (see U.S. Pat. Nos. 4,683,195 and 4,683,202). By contrast, “coupled” RT PCR methods use a common buffer optimized for reverse transcriptase and Taq DNA Polymerase activities. In one version, the annealing of reverse primer is a separate step preceding the addition of enzymes, which are then added to the single reaction vessel. In another version, the reverse transcriptase activity is a component of the thermostable Tth DNA polymerase. Annealing and cDNA synthesis are performed in the presence of Mn2+ then PCR is carried out in the presence of Mg2+ after the removal of Mn2+ by a chelating agent. Finally, the “continuous” method (e.g., one step reverse transcriptase-PCR) integrates the three reverse transcriptase-PCR steps into a single continuous reaction that avoids the opening of the reaction tube for component or enzyme addition. Continuous reverse transcriptase-PCR has been described as a single enzyme system using the reverse transcriptase activity of thermostable Taq DNA Polymerase and Tth polymerase and as a two enzyme system using AMV RT and Taq DNA Polymerase wherein the initial 65° C. RNA denaturation step may be omitted.

In certain embodiments, one or more primers may be labeled. As used herein, “label,” “detectable label,” or “marker,” or “detectable marker,” which are interchangeably used in the specification, refers to any chemical moiety attached to a nucleotide, nucleotide polymer, or nucleic acid binding factor, wherein the attachment may be covalent or non-covalent. Preferably, the label is detectable and renders the nucleotide or nucleotide polymer detectable to the practitioner of the invention. Detectable labels include luminescent molecules, chemiluminescent molecules, fluorochromes, fluorescent quenching agents, colored molecules, radioisotopes or scintillants. Detectable labels also include any useful linker molecule (such as biotin, avidin, streptavidin, HRP, protein A, protein G, antibodies or fragments thereof, Grb2, polyhistidine, Ni2+, FLAG tags, myc tags), heavy metals, enzymes (examples include alkaline phosphatase, peroxidase and luciferase), electron donors/acceptors, acridinium esters, dyes and calorimetric substrates. It is also envisioned that a change in mass may be considered a detectable label, as is the case of surface plasmon resonance detection. The skilled artisan would readily recognize useful detectable labels that are not mentioned above, which may be employed in the operation of the present invention.

One step reverse transcriptase-PCR provides several advantages over uncoupled reverse transcriptase-PCR. One step reverse transcriptase-PCR requires less handling of the reaction mixture reagents and nucleic acid products than uncoupled reverse transcriptase-PCR (e.g., opening of the reaction tube for component or enzyme addition in between the two reaction steps), and is therefore less labor intensive, reducing the required number of person hours. One step reverse transcriptase-PCR also requires less sample, and reduces the risk of contamination. The sensitivity and specificity of one-step reverse transcriptase-PCR has proven well suited for studying expression levels of one to several genes in a given sample or the detection of pathogen RNA. Typically, this procedure has been limited to use of gene-specific primers to initiate cDNA synthesis.

The ability to measure the kinetics of a PCR reaction by on-line detection in combination with these reverse transcriptase-PCR techniques has enabled accurate and precise quantitation of RNA copy number with high sensitivity. This has become possible by detecting the reverse transcriptase-PCR product through fluorescence monitoring and measurement of PCR product during the amplification process by fluorescent dual-labeled hybridization probe technologies, such as the 5′ fluorogenic nuclease assay (“TaqMan™”) or endonuclease assay (“CataCleave™”), discussed below.

### Real-Time PCR Using a CataCleave™ Probe

In other embodiments, HPV sequences are detected using Catacleave PCR. This PCR detection method employ fluorescently labeled probes that bind to the newly synthesized DNA or dyes whose fluorescence emission is increased when intercalated into double stranded DNA. Real time detection methodologies are applicable to PCR detection of target nucleic acid sequences in genomic DNA or genomic RNA.

The probes are generally designed so that donor emission is quenched in the absence of target by fluorescence resonance energy transfer (FRET) between two chromophores. The donor chromophore, in its excited state, may transfer energy to an acceptor chromophore when the pair is in close proximity. This transfer is always non-radiative and occurs through dipole-dipole coupling. Any process that sufficiently increases the distance between the chromophores will decrease FRET efficiency such that the donor chromophore emission can be detected radiatively. Common donor chromophores include FAM, TAMRA, VIC, JOE, Cy3, Cy5, and Texas Red.) Acceptor chromophores are chosen so that their excitation spectra overlap with the emission spectrum of the donor. An example of such a pair is FAM-TAMRA. There are also non fluorescent acceptors that will quench a wide range of donors. Other examples of appropriate donor-acceptor FRET pairs will be known to those skilled in the art.

Common examples of FRET probes that can be used for real-time detection of PCR include molecular beacons (e.g., U.S. Pat. No. 5,925,517), TaqMan™ probes (e.g., U.S. Pat. Nos. 5,210,015 and 5,487,972), and CataCleave™ probes (e.g., U.S. Pat. No. 5,763,181). The molecular beacon is a single stranded oligonucleotide designed so that in the unbound state the probe forms a secondary structure where the donor and acceptor chromophores are in close proximity and donor emission is reduced. At the proper reaction temperature the beacon unfolds and specifically binds to the amplicon. Once unfolded the distance between the donor and acceptor chromophores increases such that FRET is reversed and donor emission can be monitored using specialized instrumentation. TaqMan™ and CataCleave™ technologies differ from the molecular beacon in that the FRET probes employed are cleaved such that the donor and acceptor chromophores become sufficiently separated to reverse FRET.

TaqMan™ technology employs a single stranded oligonucleotide probe that is labeled at the 5′ end with a donor chromophore and at the 3′ end with an acceptor chromophore. The DNA polymerase used for amplification must contain a 5′->3′ exonuclease activity. The TaqMan™ probe binds to one strand of the amplicon at the same time that the primer binds. As the DNA polymerase extends the primer the polymerase will eventually encounter the bound TaqMan™ probe. At this time the exonuclease activity of the polymerase will sequentially degrade the TaqMan™ probe starting at the 5′ end. As the probe is digested the mononucleotides comprising the probe are released into the reaction buffer. The donor diffuses away from the acceptor and FRET is reversed. Emission from the donor is monitored to identify probe cleavage. Because of the way TaqMan™ works a specific amplicon can be detected only once for every cycle of PCR. Extension of the primer through the TaqMan™ target site generates a double stranded product that prevents further binding of TaqMan™ probes until the amplicon is denatured in the next PCR cycle.

U.S. Pat. No. 5,763,181, of which content is incorporated herein by reference, describes another real-time detection method (referred to as “CataCleave™”). CataCleave™ technology differs from TaqMan™ in that cleavage of the probe is accomplished by a second enzyme that does not have polymerase activity. The CataCleave™ probe has a sequence within the molecule which is a target of an endonuclease, such as, for example a restriction enzyme or RNAase. In one example, the CataCleave™ probe has a chimeric structure where the 5′ and 3′ ends of the probe are constructed of DNA and the cleavage site contains RNA. The DNA sequence portions of the probe are labeled with a FRET pair either at the ends or internally. The PCR reaction includes a thermostable RNase H enzyme that can specifically cleave the RNA sequence portion of a RNA-DNA duplex. After cleavage, the two halves of the probe dissociate from the target amplicon at the reaction temperature and diffuse into the reaction buffer. As the donor and acceptors separate FRET is reversed in the same way as the TaqMan™ probe and donor emission can be monitored. Cleavage and dissociation regenerates a site for further CataCleave™ binding. In this way it is possible for a single amplicon to serve as a target or multiple rounds of probe cleavage until the primer is extended through the CataCleave™ probe binding site.

Labeling of a CataCleave™ probe

The term “probe” comprises a polynucleotide that comprises a specific portion designed to hybridize in a sequence-specific manner with a complementary region of a specific nucleic acid sequence, e.g., a target nucleic acid sequence. In one embodiment, the oligonucleotide probe is in the range of 15-60 nucleotides in length. More preferably, the oligonucleotide probe is in the range of 18-30 nucleotides in length. The precise sequence and length of an oligonucleotide probe of the invention depends in part on the nature of the target polynucleotide to which it binds. The binding location and length may be varied to achieve appropriate annealing and melting properties for a particular embodiment. Guidance for making such design choices can be found in many of the references describing TaqMan™ assays or CataCleave™, described in U.S. Pat. Nos. 5,763,181, 6,787,304, and 7,112,422, of which contents are incorporated herein by reference.

In certain embodiments, the probe is “substantially complementary” to the target nucleic acid sequence.

As used herein, the term “substantially complementary” refers to two nucleic acid strands that are sufficiently complimentary in sequence to anneal and form a stable duplex. The complementarity does not need to be perfect; there may be any number of base pair mismatches, for example, between the two nucleic acids. However, if the number of mismatches is so great that no hybridization can occur under even the least stringent hybridization conditions, the sequence is not a substantially complementary sequence. When two sequences are referred to as “substantially complementary” herein, it means that the sequences are sufficiently complementary to each other to hybridize under the selected reaction conditions. The relationship of nucleic acid complementarity and stringency of hybridization sufficient to achieve specificity is well known in the art. Two substantially complementary strands can be, for example, perfectly complementary or can contain from 1 to many mismatches so long as the hybridization conditions are sufficient to allow, for example discrimination between a pairing sequence and a non-pairing sequence. Accordingly, “substantially complementary” sequences can refer to sequences with base-pair complementarity of 100, 95, 90, 80, 75, 70, 60, 50 percent or less, or any number in between, in a double-stranded region.

As used herein, a “selected region” refers to a polynucleotide sequence of a target DNA or cDNA that anneals with the RNA sequences of a probe. In one embodiment, a “selected region” of a target DNA or cDNA can be from 1, 2, 3, 4, 5, 6, 7, 8, 9, 10, 11, 12, 13, 14, 15, 16, 17, 18, 19, 20, 21, 22, 23, 24, 25 or more nucleotides in length.

As used herein, the site-specific RNase H cleavage refers to the cleavage of the RNA moiety of the Catacleave™ probe that is entirely complimentary to and hybridizes with a target DNA sequence to form an RNA:DNA heteroduplex.

As used herein, “label” or “detectable label” of the CataCleave™ probe refers to any label comprising a fluorochrome compound that is attached to the probe by covalent or non-covalent means.

As used herein, “fluorochrome” refers to a fluorescent compound that emits light upon excitation by light of a shorter wavelength than the light that is emitted. The term “fluorescent donor” or “fluorescence donor” refers to a fluorochrome that emits light that is measured in the assays described in the present invention. More specifically, a fluorescent donor provides energy that is absorbed by a fluorescence acceptor. The term “fluorescent acceptor” or “fluorescence acceptor” refers to either a second fluorochrome or a quenching molecule that absorbs energy emitted from the fluorescence donor. The second fluorochrome absorbs the energy that is emitted from the fluorescence donor and emits light of longer wavelength than the light emitted by the fluorescence donor. The quenching molecule absorbs energy emitted by the fluorescence donor.

Any luminescent molecule, preferably a fluorochrome and/or fluorescent quencher may be used in the practice of this invention, including, for example, Alexa Fluor™ 350, Alexa Fluor™ 430, Alexa Fluor™ 488, Alexa Fluor™ 532, Alexa Fluor™ 546, Alexa Fluor™ 568, Alexa Fluor™ 594, Alexa Fluor™ 633, Alexa Fluor™ 647, Alexa Fluor™ 660, Alexa Fluor™ 680, 7-diethylaminocoumarin-3-carboxylic acid, Fluorescein, Oregon Green 488, Oregon Green 514, Tetramethylrhodamine, Rhodamine X, Texas Red dye, QSY 7, QSY33, Dabcyl, BODIPY FL, BODIPY 630/650, BODIPY 6501665, BODIPY TMR-X, BODIPY TR-X, Dialkylaminocoumarin, Cy5.5, Cy5, Cy3.5, Cy3, DTPA(Eu3+)-AMCA and TTHA(Eu3+)AMCA.

In one embodiment, the 3′ terminal nucleotide of the oligonucleotide probe is blocked or rendered incapable of extension by a nucleic acid polymerase. Such blocking is conveniently carried out by the attachment of a reporter or quencher molecule to the terminal 3′ position of the probe.

In one embodiment, reporter molecules are fluorescent organic dyes derivatized for attachment to the terminal 3′ or terminal 5′ ends of the probe via a linking moiety. Preferably, quencher molecules are also organic dyes, which may or may not be fluorescent, depending on the embodiment of the invention. For example, in a preferred embodiment of the invention, the quencher molecule is fluorescent. Generally whether the quencher molecule is fluorescent or simply releases the transferred energy from the reporter by non-radiative decay, the absorption band of the quencher should substantially overlap the fluorescent emission band of the reporter molecule. Non-fluorescent quencher molecules that absorb energy from excited reporter molecules, but which do not release the energy radiatively, are referred to in the application as chromogenic molecules.

Exemplary reporter-quencher pairs may be selected from xanthene dyes, including fluoresceins, and rhodamine dyes. Many suitable forms of these compounds are widely available commercially with substituents on their phenyl moieties which can be used as the site for bonding or as the bonding functionality for attachment to an oligonucleotide. Another group of fluorescent compounds are the naphthylamines, having an amino group in the alpha or position. Included among such naphthylamino compounds are 1-dimethylaminonaphthyl-5-sulfonate, 1-anilino-8-naphthalene sulfonate and 2-p-touidinyl6-naphthalene sulfonate. Other dyes include 3-phenyl-7-isocyanatocoumarin, acridines, such as 9-isothiocyanatoacridine and acridine orange, N-(p-(2-benzoxazolyl)phenyl)maleimide, benzoxadiazoles, stilbenes, pyrenes, and the like.

In one embodiment, reporter and quencher molecules are selected from fluorescein and rhodamine dyes.

There are many linking moieties and methodologies for attaching reporter or quencher molecules to the 5′ or 3′ termini of oligonucleotides, as exemplified by the following references: Eckstein, editor, Oligonucleotides and Analogues: A Practical Approach (IRL Press, Oxford, 1991); Zuckerman et al., Nucleic Acids Research, 15: 5305-5321 (1987) (3′ thiol group on oligonucleotide); Sharma et al., Nucleic Acids Research, 19: 3019 (1991) (3′ sulfhydryl); Giusti et al., PCR Methods and Applications, 2: 223-227 (1993) and Fung et al., U.S. Pat. No. 4,757,141 (5′ phosphoamino group via Aminolink™. II available from Applied Biosystems, Foster City, Calif.) Stabinsky, U.S. Pat. No. 4,739,044 (3′ aminoalkylphosphoryl group); Agrawal et al., Tetrahedron Letters, 31: 1543-1546 (1990) (attachment via phosphoramidate linkages); Sproat et al., Nucleic Acids Research, 15: 4837 (1987) (5′ mercapto group); Nelson et al., Nucleic Acids Research, 17: 7187-7194 (1989) (3′ amino group); and the like.

Rhodamine and fluorescein dyes are also conveniently attached to the 5′ hydroxyl of an oligonucleotide at the conclusion of solid phase synthesis by way of dyes derivatized with a phosphoramidite moiety, e.g., Woo et al., U.S. Pat. No. 5,231,191; and Hobbs, Jr., U.S. Pat. No. 4,997,928.

RNase H cleavage of the Catacleave™ Probe

In certain embodiments, the Catacleave PCR reaction can include a hot start RNase H activity.

RNase H hydrolyzes RNA in RNA-DNA hybrids. First identified in calf thymus, RNase H has subsequently been described in a variety of organisms. Indeed, RNase H activity appears to be ubiquitous in eukaryotes and bacteria. Although RNase Hs form a family of proteins of varying molecular weight and nucleolytic activity, substrate requirements appear to be similar for the various isotypes. For example, most RNase Hs studied to date function as endonucleases and require divalent cations (e.g., Mg2+, Mn2+) to produce cleavage products with 5′ phosphate and 3′ hydroxyl termini.

In prokaryotes, RNase H have been cloned and extensively characterized (see Crooke, et al., (1995) Biochem J, 312 (Pt 2), 599-608; Lima, et al., (1997) J Biol Chem, 272, 27513-27516; Lima, et al., (1997) Biochemistry, 36, 390-398; Lima, et al., (1997) J Biol Chem, 272, 18191-18199; Lima, et al., (2007) Mol Pharmacol, 71, 83-91; Lima, et al., (2007) Mol Pharmacol, 71, 73-82; Lima, et al., (2003) J Biol Chem, 278, 14906-14912; Lima, et al., (2003) J Biol Chem, 278, 49860-49867; Itaya, M., Proc. Natl. Acad. Sci. USA, 1990, 87, 8587-8591). For example, E. coli RNase HII is 213 amino acids in length whereas RNase HI is 155 amino acids long. E. coli RNase HII displays only 17% homology with E. coli RNase HI. An RNase H cloned from S. typhimurium differed from E. coli RNase HI in only 11 positions and was 155 amino acids in length (Itaya, M. and Kondo K., Nucleic Acids Res., 1991, 19, 4443-4449).

Proteins that display RNase H activity have also been cloned and purified from a number of viruses, other bacteria and yeast (Wintersberger, U. Pharmac. Ther., 1990, 48, 259-280). In many cases, proteins with RNase H activity appear to be fusion proteins in which RNase H is fused to the amino or carboxy end of another enzyme, often a DNA or RNA polymerase. The RNase H domain has been consistently found to be highly homologous to E. coli RNase HI, but because the other domains vary substantially, the molecular weights and other characteristics of the fusion proteins vary widely.

In higher eukaryotes two classes of RNase H have been defined based on differences in molecular weight, effects of divalent cations, sensitivity to sulfhydryl agents and immunological cross-reactivity (Busen et al., Eur. J. Biochem., 1977, 74, 203-208). RNase HI enzymes are reported to have molecular weights in the 68-90 kDa range, be activated by either Mn2+ or Mg2+ and be insensitive to sulfhydryl agents. In contrast, RNase HII enzymes have been reported to have molecular weights ranging from 31-45 kDa, to require Mg2+ to be highly sensitive to sulfhydryl agents and to be inhibited by Mn2+ (Busen, W., and Hausen, P., Eur. J. Biochem., 1975, 52, 179-190; Kane, C. M., Biochemistry, 1988, 27, 3187-3196; Busen, W., J. Biol. Chem., 1982, 257, 7106-7108)

An enzyme with RNase HII characteristics has also been purified to near homogeneity from human placenta (Frank et al., Nucleic Acids Res., 1994, 22, 5247-5254). This protein has a molecular weight of approximately 33 kDa and is active in a pH range of 6.5-10, with a pH optimum of 8.5-9. The enzyme requires Mg2+ and is inhibited by Mn2+ and n-ethyl maleimide. The products of cleavage reactions have 3′ hydroxyl and 5′ phosphate termini.

A detailed comparison of RNases from different species is reported in Ohtani N, Haruki M, Morikawa M, Kanaya S. J Biosci Bioeng. 1999; 88(1):12-9.

Examples of RNase H enzymes, which may be employed in the embodiments, also include, but are not limited to, thermostable RNase H enzymes isolated from thermophilic organisms such as Pyrococcus furiosus, Pyrococcus horikoshi, Thermococcus litoralis or Thermus thermophilus.

Other RNase H enzymes that may be employed in the embodiments are described in, for example, U.S. Pat. No. 7,422,888 to Uemori or the published U.S. Patent Application No. 2009/0325169 to Walder, the contents of which are incorporated herein by reference.

In one embodiment, an RNase H enzyme is a thermostable RNase H with 40%, 50%, 60%, 70%, 80%, 90%, 95% or 99% homology with the amino acid sequence of Pfu RNase HII (SEQ ID NO: 60), shown below.

| (SEQ ID NO: 60) | |
|---|---|
| MKIGGIDEAG RGPAIGPLVV ATVVVDEKNI EKLRNIGVKD SKQLTPHERK NLFSQITSIA | 60 |
| DDYKIVIVSP EEIDNRSGTM NELEVEKFAL ALNSLQIKPA LIYADAADVD ANRFASLIER | 120 |
| RLNYKAKIIA EHKADAKYPV VSAASILAKV VRDEEIEKLK KQYGDFGSGY PSDPKTKKWL | 180 |
| EEYYKKHNSF PPIVRRTWET VRKIEESIKA KKSQLTLDKF FKKP | |

The homology can be determined using, for example, a computer program DNASIS-Mac (Takara Shuzo), a computer algorithm FASTA (version 3.0; Pearson, W. R. et al., Pro. Natl. Acad. Sci., 85:2444-2448, 1988) or a computer algorithm BLAST (version 2.0, Altschul et al., Nucleic Acids Res. 25:3389-3402, 1997)

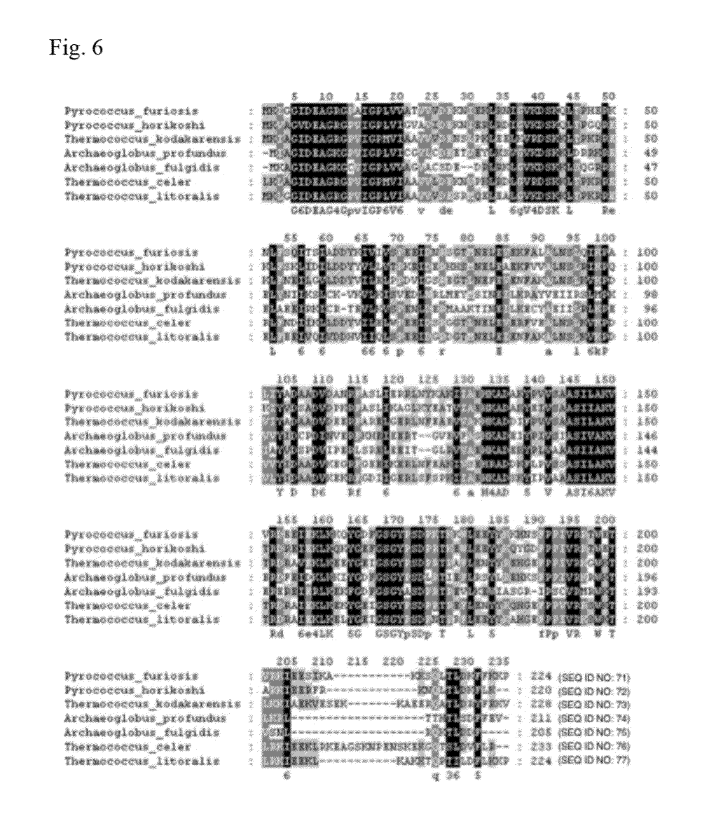

In another embodiment, an RNase H enzyme is a thermostable RNase H with at least one or more homology regions 1-4 corresponding to positions 5-20, 33-44, 132-150, and 158-173 of SEQ ID NO: 60. These homology regions were defined by sequence alignment of Pyrococcus furiosis, Pyrococcus horikoshi, Thermococcus kodakarensis, Archeoglobus profundus, Archeoglobus fulgidis, Thermococcus celer and Thermococcus litoralis RNase HII polypeptide sequences (see FIG. 6).

HOMOLOGY REGION 1:
GIDEAG RGPAIGPLVV
(SEQ ID NO: 61; corresponding to positions
5-20 of SEQ ID NO: 60)
HOMOLOGY REGION 2:
LRNIGVKD SKQL
(SEQ ID NO: 62; corresponding to positions
33-44 of SEQ ID NO: 60)
HOMOLOGY REGION 3:
HKADAKYPV VSAASILAKV
(SEQ ID NO: 63; corresponding to positions
132-150 of SEQ ID NO: 60)
HOMOLOGY REGION 4:
KLK KQYGDFGSGY PSD
(SEQ ID NO: 64; corresponding to positions
158-173 of SEQ ID NO: 60)

In one embodiment, an RNase H enzyme is a thermostable RNase H with at least one of the homology regions having 50%, 60%. 70%, 80%, 90% sequence identity with a polypeptide sequence of SEQ ID NOs: 61, 62, 63 or 64.

In another embodiment, an RNase H enzyme is a thermostable RNase H with 40%, 50%, 60%, 70%, 80%, 90%, 95% or 99% homology with the amino acid sequence of Thermus thermophilus RNase HI (SEQ ID NO: 65), shown below.

(SEQ ID NO: 65)
MNPSPRKRVA LFTDGACLGN PGPGGWAALL RFHAHEKLLS GGEACTTNNR MELKAAIEGL
KALKEPCEVD LYTDSHYLKK AFTEGWLEGW RKRGWRTAEG KPVKNRDLWE ALLLAMAPHR
VRFHFVKGHT GHPENERVDR EARRQAQSQA KTPCPPRAPT LFHEEA

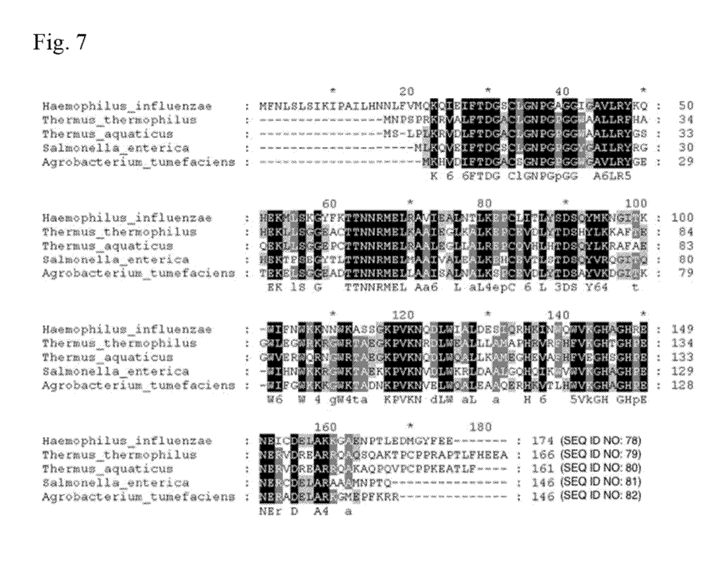

In another embodiment, an RNase H enzyme is a thermostable RNase H with at least one or more homology regions 5-8 corresponding to positions 23-48, 62-69, 117-121 and 141-152 of SEQ ID NO: 65. These homology regions were defined by sequence alignment of Haemophilus influenzae, Thermus thermophilis, Thermus acquaticus, Salmonella enterica and Agrobacterium tumefaciens RNase HI polypeptide sequences (see FIG. 7).

HOMOLOGY REGION 5:
K*V*LFTDG*C*GNPG*GG*ALLRY
(SEQ ID NO: 66; corresponding to positions
23-48 of SEQ ID NO: 65)
HOMOLOGY REGION 6:
TTNNRMEL
(SEQ ID NO: 67; corresponding to positions
62-69 of SEQ ID NO: 65)
HOMOLOGY REGION 7:
KPVKN
(SEQ ID NO: 68; corresponding to positions
117-121 of SEQ ID NO: 65)
HOMOLOGY REGION 8:
FVKGH*GH*ENE
(SEQ ID NO: 69; corresponding to positions
141-152 of SEQ ID NO: 65)

In another embodiment, an RNase H enzyme is a thermostable RNase H with at least one of the homology regions 4-8 having 50%, 60%. 70%, 80%, 90% sequence identity with a polypeptide sequence of SEQ ID NOs: 66, 67, 68 or 69.

The terms “sequence identity,” as used herein, refers to the extent that sequences are identical or functionally or structurally similar on a amino acid to amino acid basis over a window of comparison. Thus, a “percentage of sequence identity”, for example, can be calculated by comparing two optimally aligned sequences over the window of comparison, determining the number of positions at which the identical amino acid occurs in both sequences to yield the number of matched positions, dividing the number of matched positions by the total number of positions in the window of comparison (i.e., the window size), and multiplying the result by 100 to yield the percentage of sequence identity.

In certain embodiments, the RNase H can be modified to produce a hot start “inducible” RNase H.

The term “modified RNase H,” as used herein, can be an RNase H reversely coupled to or reversely bound to an inhibiting factor that causes the loss of the endonuclease activity of the RNase H. Release or decoupling of the inhibiting factor from the RNase H restores at least partial or full activity of the endonuclease activity of the RNase H. About 30-100% of its activity of an intact RNase H may be sufficient. The inhibiting factor may be a ligand or a chemical modification. The ligand can be an antibody, an aptamer, a receptor, a cofactor, or a chelating agent. The ligand can bind to the active site of the RNase H enzyme thereby inhibiting enzymatic activity or it can bind to a site remote from the RNase's active site. In some embodiments, the ligand may induce a conformational change. The chemical modification can be a cross-linking (for example, by formaldehyde) or acylation. The release or decoupling of the inhibiting factor from the RNase H may be accomplished by heating a sample or a mixture containing the coupled RNase H (inactive) to a temperature of about 65° C. to about 95° C. or higher, and/or lowering the pH of the mixture or sample to about 7.0 or lower.

As used herein, a hot start “inducible” RNase H activity can refer to the herein described modified RNase H that has an endonuclease catalytic activity that can be regulated by association with a ligand. Under permissive conditions, the RNase H endonuclease catalytic activity is activated whereas at non-permissive conditions, this catalytic activity is inhibited. In some embodiments, the catalytic activity of a modified RNase H can be inhibited at temperature conducive for reverse transcription, i.e. about 42° C., and activated at more elevated temperatures found in PCR reactions, i.e. about 65° C. to 95° C. A modified RNase H with these characteristics is said to be “heat inducible.”

In other embodiments, the catalytic activity of a modified RNase H can be regulated by changing the pH of a solution containing the enzyme.

As used herein, a “hot start” enzyme composition refers to compositions having an enzymatic activity that is inhibited at non-permissive temperatures, i.e. from about 25° C. to about 45° C. and activated at temperatures compatible with a PCR reaction, e.g. about 55° C. to about 95° C. In certain embodiment, a “hot start” enzyme composition may have a ‘hot start’ RNase H and/or a ‘hot start’ thermostable DNA polymerase that are known in the art.

Cross-linking of RNase H enzymes can be performed using, for example, formaldehyde. In one embodiment, a thermostable RNase H is subjected to controlled and limited crosslinking using formaldehyde. By heating an amplification reaction composition, which comprises the modified RNase H in an active state, to a temperature of about 95° C. or higher for an extended time, for example about 15 minutes, the cross-linking is reversed and the RNase H activity is restored.

In general, the lower the degree of cross-linking, the higher the endonuclease activity of the enzyme is after reversal of cross-linking. The degree of cross-linking may be controlled by varying the concentration of formaldehyde and the duration of cross-linking reaction. For example, about 0.2% (w/v), about 0.4% (w/v), about 0.6% (w/v), or about 0.8% (w/v) of formaldehyde may be used to crosslink an RNase H enzyme. About 10 minutes of cross-linking reaction using 0.6% formaldehyde may be sufficient to inactivate RNase HII from Pyrococcus furiosus.

The cross-linked RNase H does not show any measurable endonuclease activity at about 37° C. In some cases, a measurable partial reactivation of the cross-linked RNase H may occur at a temperature of around 50° C., which is lower than the PCR denaturation temperature. To avoid such unintended reactivation of the enzyme, it may be required to store or keep the modified RNase H at a temperature lower than 50° C. until its reactivation.

In general, PCR requires heating the amplification composition at each cycle to about 95° C. to denature the double stranded target sequence which will also release the inactivating factor from the RNase H, partially or fully restoring the activity of the enzyme.

RNase H may also be modified by subjecting the enzyme to acylation of lysine residues using an acylating agent, for example, a dicarboxylic acid. Acylation of RNase H may be performed by adding cis-aconitic anhydride to a solution of RNase H in an acylation buffer and incubating the resulting mixture at about 1-20° C. for 5-30 hours. In one embodiment, the acylation may be conducted at around 3-8° C. for 18-24 hours. The type of the acylation buffer is not particularly limited. In an embodiment, the acylation buffer has a pH of between about 7.5 to about 9.0.

The activity of acylated RNase H can be restored by lowering the pH of the amplification composition to about 7.0 or less. For example, when Tris buffer is used as a buffering agent, the composition may be heated to about 95° C., resulting in the lowering of pH from about 8.7 (at 25° C.) to about 6.5 (at 95° C.).

The duration of the heating step in the amplification reaction composition may vary depending on the modified RNase H, the buffer used in the PCR, and the like. However, in general, heating the amplification composition to 95° C. for about 30 seconds-4 minutes is sufficient to restore RNase H activity. In one embodiment, using a commercially available buffer and one or more non-ionic detergents, full activity of Pyrococcus furiosus RNase HII is restored after about 2 minutes of heating.

RNase H activity may be determined using methods that are well in the art. For example, according to a first method, the unit activity is defined in terms of the acid-solubilization of a certain number of moles of radiolabeled polyadenylic acid in the presence of equimolar polythymidylic acid under defined assay conditions (see Epicentre Hybridase thermostable RNase HI). In the second method, unit activity is defined in terms of a specific increase in the relative fluorescence intensity of a reaction containing equimolar amounts of the probe and a complementary template DNA under defined assay conditions.

### Attachment of a CataCleave™ Probe to a Solid Support

In one embodiment, the oligonucleotide probe can be attached to a solid support. Different probes may be attached to the solid support and may be used to simultaneously detect different target sequences in a sample. Reporter molecules having different fluorescence wavelengths can be used on the different probes, thus enabling hybridization to the different probes to be separately detected.

Examples of preferred types of solid supports for immobilization of the oligonucleotide probe include controlled pore glass, glass plates, polystyrene, avidin coated polystyrene beads cellulose, nylon, acrylamide gel and activated dextran, controlled pore glass (CPG), glass plates and high cross-linked polystyrene. These solid supports are preferred for hybridization and diagnostic studies because of their chemical stability, ease of functionalization and well defined surface area. Solid supports such as controlled pore glass (500 Å, 1000 Å) and non-swelling high cross-linked polystyrene (1000 Å) are particularly preferred in view of their compatibility with oligonucleotide synthesis.

The oligonucleotide probe may be attached to the solid support in a variety of manners. For example, the probe may be attached to the solid support by attachment of the 3′ or 5′ terminal nucleotide of the probe to the solid support. However, the probe may be attached to the solid support by a linker which serves to distance the probe from the solid support. The linker is most preferably at least 30 atoms in length, more preferably at least 50 atoms in length.

Hybridization of a probe immobilized to a solid support generally requires that the probe be separated from the solid support by at least 30 atoms, more-preferably at least 50 atoms. In order to achieve this separation, the linker generally includes a spacer positioned between the linker and the 3′ nucleoside. For oligonucleotide synthesis, the linker arm is usually attached to the 3′-OH of the 3′ nucleoside by an ester linkage which can be cleaved with basic reagents to free the oligonucleotide from the solid support.

A wide variety of linkers are known in the art which may be used to attach the oligonucleotide probe to the solid support. The linker may be formed of any compound which does not significantly interfere with the hybridization of the target sequence to the probe attached to the solid support. The linker may be formed of a homopolymeric oligonucleotide which can be readily added on to the linker by automated synthesis. Alternatively, polymers such as functionalized polyethylene glycol can be used as the linker. Such polymers are preferred over homopolymeric oligonucleotides because they do not significantly interfere with the hybridization of probe to the target oligonucleotide. Polyethylene glycol is particularly preferred because it is commercially available, soluble in both organic and aqueous media, easy to functionalize, and completely stable under oligonucleotide synthesis and post-synthesis conditions.

The linkages between the solid support, the linker and the probe are preferably not cleaved during removal of base protecting groups under basic conditions at high temperature. Examples of preferred linkages include carbamate and amide linkages. Immobilization of a probe is well known in the art and one skilled in the art may determine the immobilization conditions.

According to one embodiment of the method, the CataCleave™ probe is immobilized on a solid support. The CataCleave™ probe comprises a detectable label and DNA and RNA nucleic acid sequences, wherein the probe's RNA nucleic acid sequences are entirely complementary to a selected region of the target DNA sequence and the probe's DNA nucleic acid sequences are substantially complementary to DNA sequences adjacent to the selected region of the target DNA sequence. The probe is then contacted with a sample of nucleic acids in the presence of RNase H and under conditions where the RNA sequences within the probe can form a RNA:DNA heteroduplex with the complementary DNA sequences in the PCR fragment. RNase H cleavage of the RNA sequences within the RNA:DNA heteroduplex results in a real-time increase in the emission of a signal from the label on the probe, wherein the increase in signal indicates the presence of the target DNA sequence.

According to another embodiment of the method, the CataCleave™ probe, immobilized on a solid support, comprises a detectable label and DNA and RNA nucleic acid sequences, wherein the probe's RNA nucleic acid sequences are entirely complementary to a selected region of the target DNA sequence and the probe's DNA nucleic acid sequences are substantially complementary to DNA sequences adjacent to the selected region of the target DNA sequence. The probe is then contacted with a sample of nucleic acids in the presence of RNase H and under conditions where the RNA sequences within the probe can form a RNA:DNA heteroduplex with the complementary DNA sequences in the PCR fragment. RNase H cleavage of the RNA sequences within the RNA:DNA heteroduplex results in a real-time increase in the emission of a signal from the label on the probe.

Immobilization of the probe to the solid support enables the target sequence hybridized to the probe to be readily isolated from the sample. In later steps, the isolated target sequence may be separated from the solid support and processed (e.g., purified, amplified) according to methods well known in the art depending on the particular needs of the researcher.

### Kits

The disclosure herein also provides for a kit format which comprises a package unit having one or more reagents for the real time PCR detection of high risk HPV target nucleic acid sequences. The kit may also contain one or more of the following items: buffers, instructions, and positive or negative controls. Kits may include containers of reagents mixed together in suitable proportions for performing the methods described herein. Reagent containers preferably contain reagents in unit quantities that obviate measuring steps when performing the subject methods.

Kits may also contain reagents for real-time PCR including, but not limited to, a hot start composition comprising a thermostable nucleic acid polymerase, a hot start thermostable RNase H, the primers described herein that can amplify a high risk HPV target nucleic acid sequence, a fluorescent dye and/or a labeled CataCleave™ oligonucleotide probe that anneals to the real-time PCR product and allows for the quantitative detection of the target nucleic acid sequence according to the methodology described herein.

In another embodiment, the kit reagents further comprised reagents for the extraction of genomic DNA or RNA from a biological sample. Kit reagents may also include reagents for reverse transcriptase-PCR analysis where applicable.

Any patent, patent application, publication, or other disclosure material identified in the specification is hereby incorporated by reference herein in its entirety. Any material, or portion thereof, that is said to be incorporated by reference herein, but which conflicts with existing definitions, statements, or other disclosure material set forth herein is only incorporated to the extent that no conflict arises between that incorporated material and the present disclosure material.

### EXAMPLES

The present invention will be described in further detail with reference to the following examples. These examples are for illustrative purposes only and are not intended to limit the scope of the invention.

### Example 1

### SYBR Green I Real-Time PCR Detection of High Risk HPV

Real-time reactions combined 2 uL of DNA template and 23 uL of PCR reaction mix. The PCR reaction mix contained 32 mM HEPES ((4-(2-hydroxyethyl)-1-piperazineethanesulfonic acid)-KOH, pH 7.8, 50 mM potassium acetate, 6 mM magnesium acetate, 0.11% bovine serum albumin, 1% dimethylsulfoxide, 120 nM forward primer, 120 nM reverse primer, dUTP/NTP mix (80 uM dGTP, dCTP, dATP and 160 uM dUTP), 2.5 Units Thermus aquaticus DNA polymerase, 1 μL of diluted SYBR Green I dye, and 1 Unit Uracil-N-Glycosylase.

Plasmid template of each HPV genotype was synthesized by IDT. A dilution of purified plasmid was tested from 10 copies to 10⁶ copies per reaction. A total of 14 genotypes of high risk HPV were tested.

Reactions were assembled at room temperature and run on a Roche Lightcycler 480 using the following cycling protocol:

37° C. for 5 minutes;

95° C. for 10 minutes;

then 5 cycles of 1st-stage amplification,

95° C. for 10 seconds,

48° C. for 60 seconds, and

72° C. for 30 seconds;

then 50 cycles of 2nd-stage amplification,

95° C. for 10 seconds,

50° C. for 60 seconds, and

72° C. for 30 seconds.

Emission of SYBR Green I dye fluorescence was monitored during the 72° C. step.

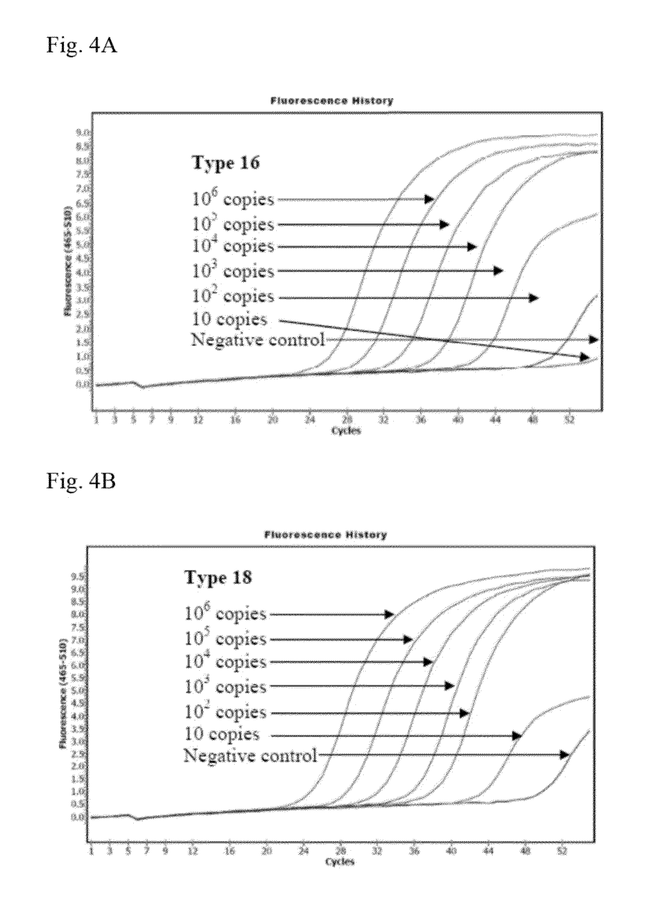

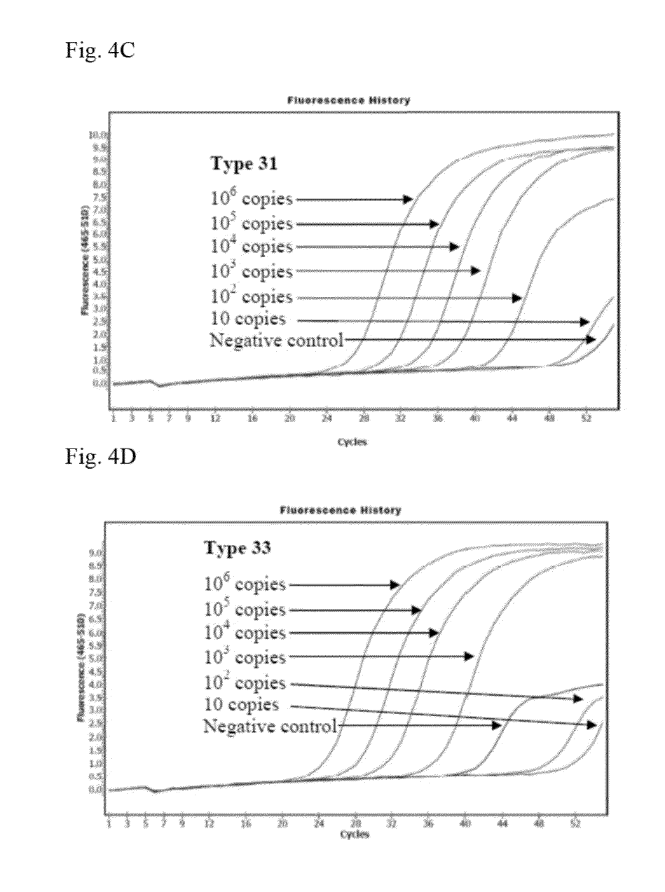

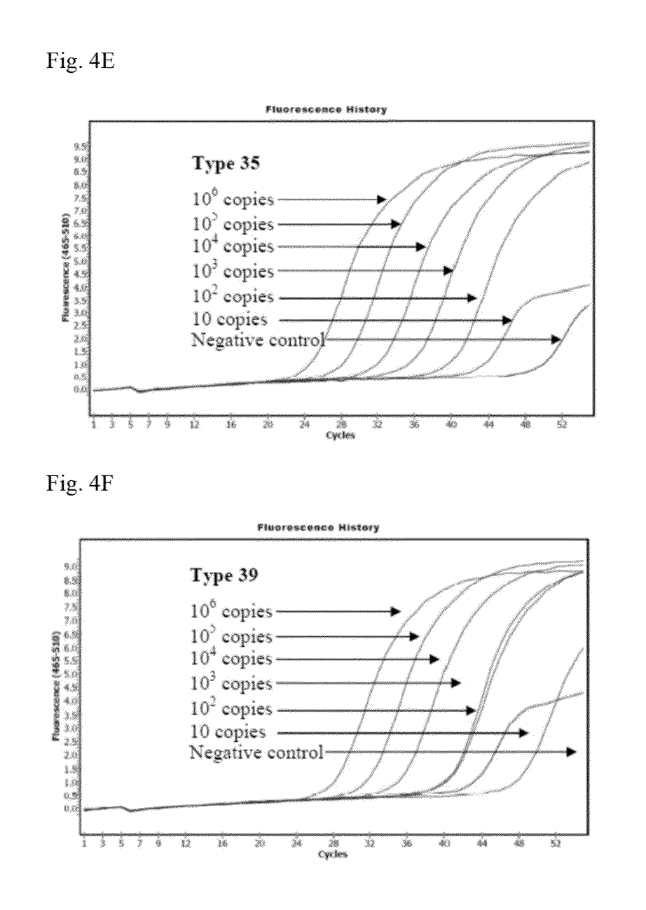

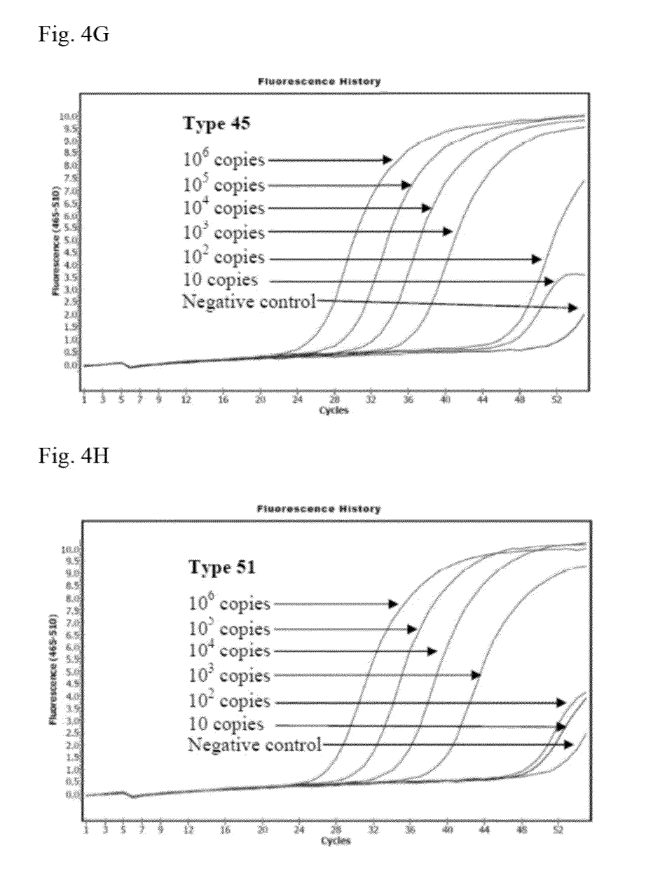

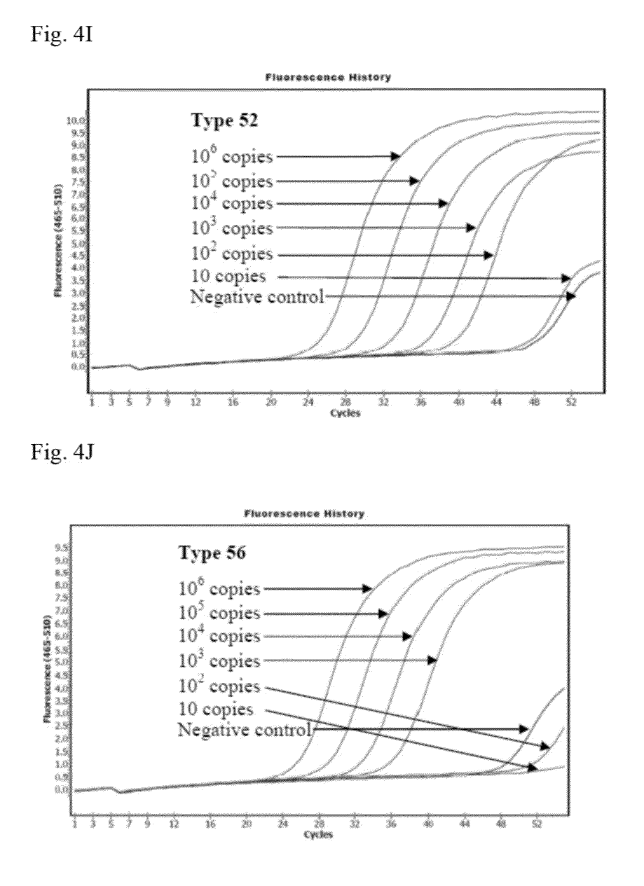

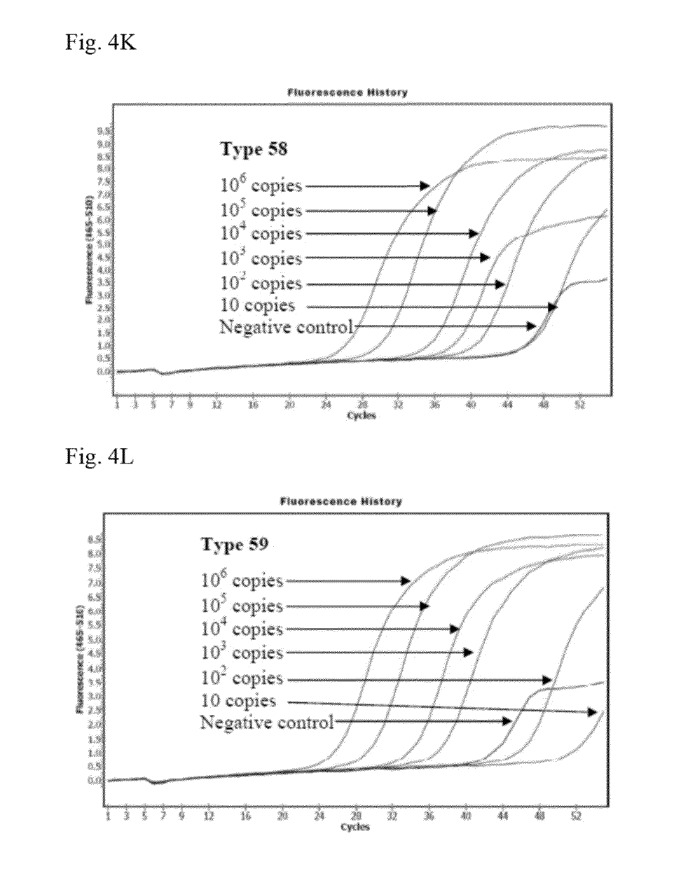

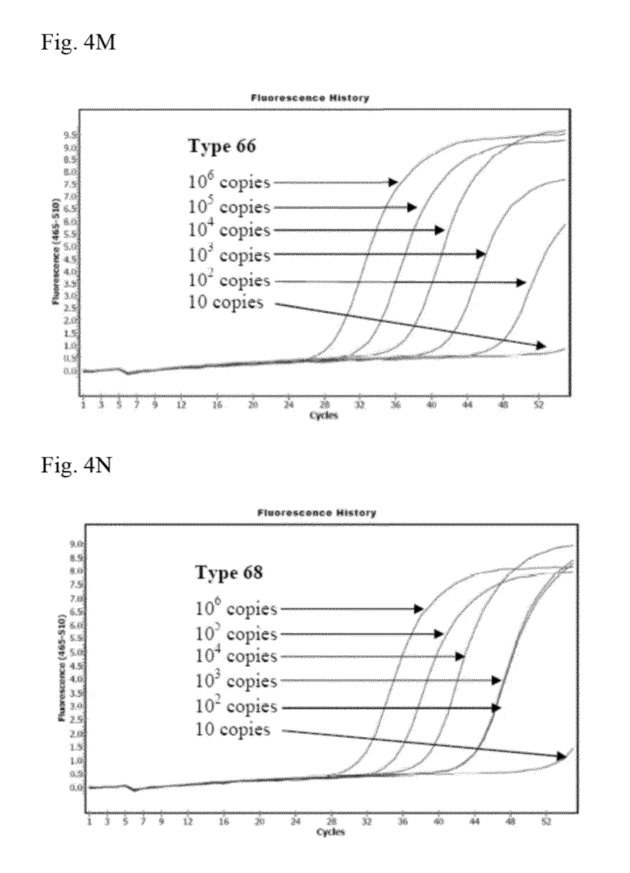

A total of 14 high risk HPV genotypes (i.e. HPV genotypes 16, 18, 31, 33, 35, 39, 45, 51, 52, 56, 58, 59, 66 and 68) were tested using purified plasmid DNA. For each genotype, a dilution ranging from 10 copies to 10⁶ copies of plasmid DNA per reaction was tested (see FIG. 4).

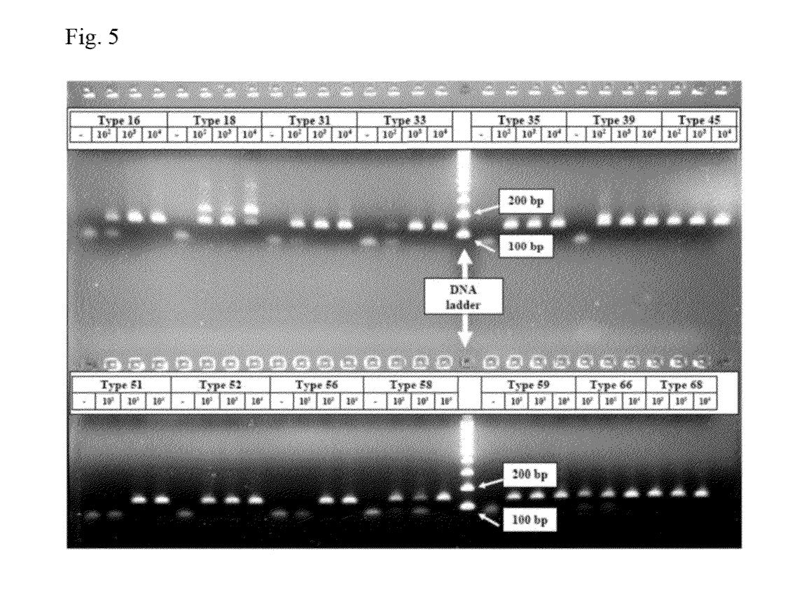

Due to high heterogeneity amongst HPV genotypes, the annealing temperature was lowered to 48-50° C. for 1 minute per cycle. This however favored formation of primer dimers. As shown in the amplification curves depicted in FIG. 4, most negative controls formed primer dimer at around cycle 40. Therefore, in addition to melting curve analysis, all PCR reactions including the negative control, the PCR amplification of each HPV genotype at dilutions of 100 copies, 1,000 copies and 10,000 copies were subsequently analyzed by gel electrophoresis (FIG. 5) to confirm sequence-specific amplification of the HPV target nucleic acid sequence, the expected amplicon size being about 140 bp.

As shown in the real-time PCR and gel electrophoresis results, the HPV assay was able to detect about 100 copies of HPV genotypes 16, 18, 31, 33, 35, 39, 45, 52, 58, 59, 66, 68 and about 1000 copies of HPV genotype 51.

1. A population of Human Papillomavirus (HPV)-specific oligonucleotides each having a nucleotide sequence that aligns with any one of the HPV nucleotide sequences of SEQ ID NOs: 31-55,
wherein each oligonucleotide within said population comprises at least 10 consecutive nucleotides selected from the nucleotide sequence of
(SEQ ID NO: 56)
GGTAGATACTACHMGYAGYAC,
wherein H is A or C or T/U, Y is C or T/U and M is A or C, and
wherein said oligonucleotides are less than about 35 nucleotides in length.

2. The population of Human Papillomavirus (HPV)-specific oligonucleotides according to claim 1,
wherein each oligonucleotide within said population comprises the sequence
(SEQ ID NO: 57)
TACHMGYAGYAC,
wherein H is A or C or T/U, Y is C or T/U and M is A or C.

3. A population of Human Papillomavirus (HPV)-specific oligonucleotides each having a nucleotide sequence that aligns with the complementary nucleotide sequence of any one of the HPV nucleotide sequences of SEQ ID NOs: 31-55,
wherein each oligonucleotide within said population comprises at least 10 consecutive nucleotides selected from the nucleotide sequence of TGTAAATCATAYT (SEQ ID NO: 58),
wherein Y is C or T/U, and
wherein said oligonucleotides are less than about 35 nucleotides in length.

4. The population of HPV-specific primers according to claim 1,
wherein each oligonucleotide within said population comprises the sequence
(SEQ ID NO: 59)
AATCAATCATAYT,
wherein H is A or C or T/U, Y is C or T/U and M is A or C, and

5. A kit for the simultaneous real-time PCR detection of high risk Human Papillomavirus (HPV) genotypes comprising a forward amplification primer having the nucleotide sequence of SEQ ID NO: 1.

6. A kit for the real-time PCR detection of high risk Human Papillomavirus (HPV) genotypes comprising a reverse amplification primer having the nucleotide sequence of SEQ ID NO: 16.

7. The kit of claim 5, further comprising a DNA and/or RNA dependent DNA polymerase activity.

8. A method for the real-time detection of high risk Human Papillomavirus (HPV) genotypes in a sample, comprising the steps of:
providing a sample to be tested for the presence of high risk HPV genotype DNA;
providing a forward amplification primer having the nucleotide sequence of SEQ ID NO: 1 and a reverse amplification primer having the nucleotide sequence of SEQ ID NO: 16, wherein said forward and reverse primers simultaneously anneal to target HPV DNA sequences;
amplifying a PCR fragment between the forward and reverse amplification primers in the presence of an amplification buffer comprising an amplifying polymerase activity and a fluorescent dye, and
detecting a real-time increase in the emission of a fluorescent signal, wherein the increase in the fluorescent signal indicates the presence of one or more high risk HPV genotypes in said sample.

9. The method of claim 8, wherein said high risk HPV genotypes comprise HPV genotypes 16, 18, 31, 33, 35, 39, 45, 51, 52, 56, 58, 59, 66 and 68.

10. The method of claim 8, wherein said HPV target DNA sequences comprise the nucleotide sequences of SEQ ID NOs: 31-55.

11. The method of claim 7, wherein said increase in the fluorescent signal can detect the presence of about 100 copies of HPV DNA from genotypes 16, 18, 31, 33, 35, 39, 45, 52, 58, 59, 66 and 68 and about 1,000 copies of HPV DNA from genotype 51.

12. The method of claim 7, wherein the amplifying polymerase activity is an activity of a thermostable DNA polymerase.

13. The method of claim 7, wherein said fluorescent dye is SYBR™ Green I.

14. The method of claim 7, wherein the PCR fragment is linked to a solid support.

15. The method of claim 18, wherein the nucleic acid within the sample is pre-treated with uracil-N-glycosylase.

16. A method for the real-time PCR detection of high risk HPV in a sample, comprising the steps of:
providing a sample to be tested for the presence of high risk HPV genotype RNA;
providing a forward amplification primer having the nucleotide sequence of SEQ ID NO: 1 and a reverse amplification primer having the nucleotide sequence of SEQ ID NO: 16, wherein said forward and reverse primers simultaneously anneal to target HPV nucleic acid sequences;
reverse transcribing high risk HPV RNAs in the presence of a reverse transcriptase buffer comprising reverse transcriptase activity and the reverse amplification primer to produce a target a high risk HPV cDNA sequence;
amplifying a PCR fragment between the forward and reverse amplification primers in the presence of the target HPV cDNA sequence and an amplification buffer comprising an amplifying polymerase activity and a fluorescent dye, and
detecting a real-time increase in the emission of a fluorescent signal,
wherein the increase in the fluorescent signal indicates the presence of one or more high risk HPV genotypes in said sample.

17. The method of claim 16, wherein said high risk HPV genotypes comprises HPV genotypes 16, 18, 31, 33, 35, 39, 45, 51, 52, 56, 58, 59, 66 and 68.

18. The method of claim 16, wherein said HPV target DNA sequences comprise the nucleotide sequences of SEQ ID NOs: 31-55.

19. The method of claim 16, wherein said increase in the fluorescent signal can detect the presence of about 100 copies of HPV DNA from genotypes 16, 18, 31, 33, 35, 39, 45, 52, 58, 59, 66 and 68 and about 1,000 copies of HPV DNA from genotype 51.

20. The kit of claim 6, further comprising a DNA and/or RNA dependent DNA polymerase activity.
